(19) United States
(12) Patent Application Publication
(10) Pub. No.: US 2017/0109499 A1
(43) Pub. Date: Apr. 20, 2017

(54) DISEASE MANAGEMENT SYSTEMS COMPRISING DIETARY SUPPLEMENTS

(71) Applicants: Rajiv DOSHI, Los Altos, CA (US); Michael L. FAVET, San Jose, CA (US)

(72) Inventors: Rajiv DOSHI, Los Altos, CA (US); Michael L. FAVET, San Jose, CA (US)

(21) Appl. No.: 15/297,072

(22) Filed: Oct. 18, 2016

Related U.S. Application Data
(60) Provisional application No. 62/243,378, filed on Oct. 19, 2015.

(51) Int. Cl. G06F 19/00; H04L 29/06; A61K 31/56

(52) U.S. Cl. CPC G06F 19/3475; A61K 31/56; G06F 19/325; H04L 63/0428; G06F 19/3431

(57) ABSTRACT

Described herein are integrated programs and systems that manage or treat chronic conditions such as elevated cholesterol or elevated blood pressure and other health conditions. The integrated system may include a combination of: a dietary supplement; behavior change messaging through packaging (including use of unit dose packaging) and/or package inserts; video content available through the web or mobile means; interactive discussions with healthcare providers; and an app or website that supports behavior change as well as tracks ongoing compliance to the dietary supplement and behavior change as well as physiologic endpoints. The integrated system may thus create a routine set of behaviors for the user to help manage or treat various health conditions.

### CROSS REFERENCE TO RELATED APPLICATIONS

This application claims priority to U.S. Provisional Patent Application No. 62/243,378, filed on Oct. 19, 2015 which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The systems and methods described herein relate to integrated programs to manage chronic diseases and other health conditions comprising the combination of dietary supplements, packaging providing educational or motivational content, video and webinar based education and/or interactive apps or websites to promote, track and incent certain healthy behaviors. In many cases, the combination of one or more of these components into an integrated system produces additive and clinically important health benefits which may find benefit in corporate health and other clinical settings.

### BACKGROUND

The management of chronic medical and other health conditions, including various disease states and pre-disease states can be difficult, as successful treatment or health maintenance frequently needs to be done by the patient or individual on an ongoing basis. Healthcare providers generally have limited time to spend with their patients and effectively educating patients about behavior change may not always be possible. Further, the patient or individual may not be aware of the importance of treating various conditions (to prevent longer term health consequences for example) and may not be aware of the actual strategies or interventions that should be performed or maintained on a daily basis. It has been said that patients need to see the same (or similar) educational messages at least three times before they learn a new behavior and then make it routine.

Various solutions that seek to improve health and wellness include, but are not limited to behavior change or modification approaches including diet and exercise, medication, dietary supplements, personal or other coaching whether in person or remote (online, phone, etc.), health and wellness apps or interactive websites, educational products such as videos, online content, medical literature and handouts, etc. Each solution may have its advantages and drawbacks. The goal of all of these solutions whether used individually or in combination is to provide sustained health impact for the user, as in many cases, the health conditions are chronic in nature and ongoing adherence to the programs is essential to prevent long term sequelae or otherwise optimize health and wellness. Part of this goal is to create a routine of certain behaviors that the user does every day.

Common chronic conditions include but are not limited to gastroesophageal reflux disease (GERD), acne, allergy, attention deficit hyperactivity disorder, altitude sickness, Alzheimer's disease, anorexia nervosa, arthritis, Asperger syndrome, asthma, autism, back pain, alopecia and other hair and skin disorders, bipolar disorder, body dysmorphic disorder, traumatic brain injury, acute bronchitis, cancer, canker sores, carpal tunnel syndrome, celiac disease, cervical cancer, elevated cholesterol and other lipid disorders, COPD, heart failure, inflammatory bowel disease, dandruff, deep vein thrombosis, depression, diabetes mellitus, diverticulitis, drug abuse, dysfunctional uterine bleeding, dyslexia, ear infections, eating disorders, dermatitis, endometriosis, benign prostatic hyperplasia, epilepsy, erectile dysfunction, eye problems, fibromyalgia, gallbladder disease, gallstones, anxiety disorders, herpes simplex, glomerulonephritis and other kidney disease, gout, gum and dental diseases, headache, deafness and other hearing disorders, myocardial infarction and coronary artery disease, cardiovascular disease, heartburn, hemorrhoids, hepatitis and other liver disease, herniated disc and other spine pain, hiatal hernia, AIDS/HIV, hives, hyperglycemia, hyperkalemia, hypertension and prehypertension, hypotension, hyperthyroidism, hypothyroidism, infectious diseases, influenza, infertility, iron deficiency anemia, joint pain, nephropathy, malaria, memory loss, menopause, migraine, muscle cramps, muscle fatigue/pain, neck pain, obesity, osteoarthritis, osteomyelitis, osteoporosis, polycystic ovary syndrome, pain, Parkinson's disease, peripheral vascular disease, peptic ulcer, post-nasal drip, posttraumatic stress disorder, premenstrual syndrome, psoriasis. renal disease, restless legs syndrome, rheumatoid arthritis, rheumatic fever, rosacea, sciatica, personality disorders, schizophrenia, sexually transmitted disease, sinusitis and rhino sinusitis, skin rash, sleep disorders, snoring, anxiety disorder, infection, pharyngitis, systemic lupus erythematosus, tooth decay, tuberculosis, ulcers, urinary tract infection, varicose veins. Any or all of these conditions can be treated with the integrated systems described herein.

Cholesterol and Lipid Disorders

Elevated cholesterol and other lipid disorders are one example of a common chronic medical condition that healthcare providers and patients are interested in treating or managing. In many cases, the goal of management is to keep the patient's lipid profile within normal limits. In other cases, the goal is to improve or treat abnormal or borderline abnormal lipid profiles, with the goal of preventing heart disease or heart attacks.

The serum lipid profile is used to assess the risk an individual has for cardiovascular disease. Among the various parameters measured are the levels of triglycerides, total cholesterol, LDL-cholesterol, and HDL-cholesterol. Low density lipoproteins (LDL) are considered to be the unhealthy type of cholesterol, whereas high density lipoproteins (HDL) are considered to be a healthy type of cholesterol. While cholesterol is essential for cell membranes in addition to being a precursor for bile acid and steroid hormone synthesis, it is poorly soluble in blood and requires the assistance of transport molecules. Lipoproteins provide this function to act as vehicles for the transport of cholesterol.

HDL and LDL differ in size and density. HDL is the smallest lipoprotein and is largely involved in the removal of excess cholesterol, which may be disposed of in the liver. LDL on the other hand is larger than HDL and serves as the main transporter of cholesterol within the blood. Blood transports cholesterol to cells for use, including the arteries, where high levels of cholesterol may lead to the formation of plaques resulting in cardiovascular disease. One of the most accurate and accepted predictors of health measures is the HDL/LDL ratio. Body weight reduction, through dieting, has been shown to favorably change this ratio. Medications such as statins also work very well to reduce LDL levels and prevent progression to coronary artery disease (and heart attacks).

However, in spite of the proven efficacy of prescription medications such as statins, many patients (especially younger ones) are reluctant to take prescription medications on an ongoing basis. Many patients fear the various complications that can come from taking statin medications. These include muscle pain and damage to the liver. As a result, these patients are looking for non-drug options which may include different approaches to behavior change (such as diet and exercise) or may include dietary supplements.

One of the most well studied and recommended dietary supplements for use in cholesterol management is plant sterols or plant stanols (also known as phytosterols). Plant sterols (and stanols which are related compounds—plant sterols and stanols are used interchangeably in this application) improve blood cholesterol levels by inhibiting cholesterol absorption, decreasing total blood cholesterol levels, decreasing blood LDL levels and decreasing triglyceride levels. Plant sterols are structurally similar to cholesterol and when ingested by humans have been found to reduce cholesterol absorption and serum cholesterol levels, while not being absorbed themselves. Lowering of circulating cholesterol and low-density lipoprotein cholesterol is an important part of a strategy to prevent and treat cardiovascular disease and especially coronary heart disease according to the National Cholesterol Education Program which is a widely accepted set of clinical practice guidelines in the United States.

Cholesterol absorption in the intestines is a critical component of whole body cholesterol metabolism. Cholesterol derived from the diet and also from endogenous biliary secretion enters the intestine and approximately 50% of the mixed intestinal load is absorbed. The failure to absorb cholesterol quantitatively is therefore a key mechanism for the elimination of cholesterol from the body. Plant sterols block this absorption representing an effective and safe means of reducing cholesterol levels. Since phytosterols are natural products which are non-toxic and inexpensive byproducts of food processing, they may be important in the treatment of individuals with mildly increased serum cholesterol or for the general population in food products or dietary supplements. The use of phytosterols could reduce the need for systemically absorbed drugs or could enable patients to reduce their dosages of prescription medications.

One of the challenges with currently available plant sterol products is compliance. Many patients do not like taking large pills that can get stuck in their throat, causing gagging or discomfort. Other food based products containing plant sterols such as margarine or juice are impractical to have on an ongoing basis or have excessive fat and/or sugar which patients and healthcare providers seek to avoid. Thus alternative preparations containing plant sterols are required, preparations that drive compliance and therefore ongoing clinical efficacy. This issue of compliance is particularly important for plant sterols as they need to be taken with meals (or at least around meal time) for at least two meals per day to maximize clinical effect.

Further it should be noted that plant sterols are part of the major medical societies' recommendations and are generally recommended in concert with other behavior change modifications including diet and exercise. For example, plant sterols are included within the “TLC” diet guidelines promoted by the US NIH. To maximize clinical efficacy, it would be ideal to combine the benefits of plant sterols with the benefits of behavior change. Indeed the combination is expected to lead to synergistic benefits.

Hypertension and Prehypertension

Worldwide, approximately 1 billion people are affected by hypertension (high blood pressure) and 7 million deaths per year are attributed to hypertension. In the United States, ˜67 million adults have high blood pressure. For every 20 mmHg increase in systolic blood pressure and 10 mmHg increase in diastolic blood pressure, the risk of cardiovascular disease morbidity doubles. Furthermore, in the United States, another ˜65 million adults are affected by pre-hypertension (mildly elevated blood pressure) which itself is associated with morbidity and mortality. These numbers are expected to significantly increase in the coming years, as obesity rates continue to increase and the definitions of hypertension/prehypertension are adjusted (leading more people to be included).

Guidelines recommend screening every two years for persons with systolic and diastolic pressures below 120 mmHg and 80 mmHg, respectively (normal blood pressure), and yearly for persons with a systolic pressure of 120 to 139 mmHg or a diastolic pressure of 80 to 89 mmHg (pre-hypertension). A physician generally makes the diagnosis of hypertension. Blood pressure measurements fall into four categories: Normal blood pressure is below 120/80 mm Hg. However, some doctors recommend 115/75 mm Hg as a better goal. Once blood pressure rises above 115/75 mm Hg, the risk of cardiovascular disease increases. Pre-hypertension is a systolic pressure between 120-139 mm Hg or a diastolic pressure between 80-89 mm Hg. Pre-hypertension tends to worsen over time. Stage 1 hypertension is a systolic pressure between 140-159 mm Hg or a diastolic pressure between 90-99 mm Hg. More severe hypertension, stage 2 hypertension is a systolic pressure of 160 mm Hg or higher or a diastolic pressure of 100 mm Hg or higher.

The diagnosis of hypertension is frequently not immediate. The physician generally takes two to three blood pressure readings each at three or more appointments over a span of weeks or months before making the diagnosis of high blood pressure. This is because blood pressure normally varies throughout the day. The physician may recommend recording blood pressure at home to provide additional information. If a patient has high blood pressure, the doctor will review the patient's medical history and conduct a physical examination.

Despite the importance of treatment, only ˜50% of persons with hypertension have their blood pressure under control, defined as a level below 140/90 mmHg (CDC). There are numerous reasons for low rates of blood pressure control, including poor access to health care and medications and lack of adherence with long-term therapy for a condition that is usually asymptomatic. Treatment of hypertension generally does not include drug therapy initially. Rather, moderate dietary sodium restriction, weight reduction in the obese, reducing alcohol intake, and regular aerobic exercise are recommended, collectively known as “behavioral modification.” Behavioral modification involves little or no risk. Patients with pre-hypertension, but without co-morbidities are treated with non-pharmacologic therapies such as behavioral modification. They should also have their blood pressure measured at least annually, or more frequently if home monitoring is available, since they are at risk of developing hypertension.

Drug therapy is often associated with side effects, some of which may actually increase coronary risk. Many patients and providers want to avoid taking drugs for the rest of the patient's lifetime. Indeed, with a vast number of patients with hypertension and pre-hypertension, it is not surprising that alternative therapies are commonly used. As an example, approximately 50% of patients with hypertension have used garlic as a treatment (Journal of Complementary and Internal Medicine 2008).

Thus, there exists a large need for non-prescription medication-based approaches to treat or manage chronic medical conditions such as elevated cholesterol, elevated blood pressure and other conditions. In other cases, there is a need for non-medical based approaches to help users maintain their current normal health status and in some cases prevent or reduce the likelihood of progression to pre-disease or disease.

Management of such chronic conditions is of particular interest to self-insured employers and employers who manage health and wellness programs. Generally, it is accepted that is it financially beneficial for both the employer and employee to keep employees healthy (by managing or preventing significant medical conditions) versus letting these conditions going undertreated or untreated. This is due to the combined negative and additive financial consequences of direct and indirect medical claims, absenteeism, presenteeism, recruiter costs, affects on achieving project timelines, etc. As a result, there is significant interest in creating corporate health and wellness programs that keep employees healthy and productive.

### SUMMARY

The present invention is directed at integrated systems that manage or treat chronic conditions such as elevated cholesterol or elevated blood pressure that require ongoing involvement of the user. The integrated system may include a combination of two or more of: a dietary supplement; behavior change messaging through packaging (including use of education/instruction on unit dose packaging or package inserts); educational or motivational video content available through the web or other means; and an interactive app or website that supports behavior change as well as tracks (and in some cases incents) compliance to the dietary supplement and behavior change activities. The integrated system creates a routine consisting of healthy behaviors, the combination of behaviors that creates synergistic and clinical effective outcomes. Further, the various components and their features are meant to interact with each other, again creating a synergistic benefit from this integrated system. Additionally, the integrated systems comprising dietary supplements may be part of larger corporate health and wellness initiatives that provide incentives to employees.

For example, described herein are integrated disease management systems for enhancing a user's health that may include: an outer box; a six or more pouches within the outer box; a dietary supplement within each of the pouches; wherein each pouch comprises a front, having a first panel, and a back having a second panel, and wherein the first panel of each pouch includes an educational message that is different from the educational message on any of the other pouches.

Any of these systems may include a non-transitory computer-readable storage medium storing a set of instructions capable of being executed by a smartphone, that, when executed by the smartphone, causes the smartphone to: receive input of user activities including user consumption of a dietary supplement and one or more of: user physical activity, and user food consumption; score the inputted user activities; track scores over time; and display one or more representations of user progress based on the scores. The non-transitory computer-readable storage medium may be further configured to transmit user scores to a remote server. For example, a remote server may create customized content based on received input of user activities and transmits the customized content to the smartphone for display to the user.

Any of these systems may also include a remote server executing a second set of instructions capable of being executed by a processor of the remote server, the second set of instructions that, when executed by the processor, causes the server to receive user scores and to indicate distribution of a reward to a user based on the received user scores.

Any of these systems described herein may also include a library of videos directed to health and wellness topics, further wherein the set of instructions causes the smartphone to access the library of videos for display to the user.

In general, these dietary supplements may include a pill, tablet, gummy, powder, softgel, chew, capsule, or gel. In any of the systems and methods described herein, the dietary supplement may include a phytosterol.

The pouches may include one, two or more chambers. For example, a pouch may include at least two sealed chambers, wherein each chamber includes the dietary supplement.

In any of the variations described herein, the pouch may include a hidden message that is revealed only by opening the pouch. For example, the pouch may include a message (e.g., related to the supplement, or the condition being addressed by the supplement) that is revealed by tearing open the supplement (e.g., within an inner region of the pouch). The back of the pouch may comprise the second panel and a third panel, wherein the second and third panels are adjacent to each other when the pouch is closed, and a fourth panel between the second and third panels that is exposed only when the pouch is opened, to reveal hidden text or image related to the educational message.

Also described herein are non-transitory computer-readable storage medium storing a set of instructions capable of being executed by a smartphone, that, when executed by the smartphone, causes the smartphone to: receive input of user activities including: user consumption of a dietary supplement, user physical activity, and user food consumption; score the inputted user activities; track scores over time; and display one or more representations of user progress based on the scores. In general, a non-transitory computer-readable storage medium may include software, hardware, firmware, a dedicated electronic memory, a readable/writable media, etc.

As mentioned, any of these systems may include a remote server executing a second set of instructions capable of being executed by a processor of the remote server, the second set of instructions that, when executed by the processor, causes the server to receive user scores and to indicate distribution of a reward to a user based on the received user scores.

Also described herein are integrated disease management systems for enhancing a user's health. A system may include: an outer box; a plurality of pouches within the outer box; a dietary supplement within each of the plurality of pouches; wherein each pouch comprises a front, having a first panel, and a back having a second panel, wherein the first panel includes an educational message and wherein the second panel includes one or more images corresponding to the educational message; and a non-transitory computer-readable storage medium storing a set of instructions capable of being executed by a smartphone, that, when executed by the smartphone, causes the smartphone to: receive input of user activities including: user consumption of the dietary supplement, user physical activity, and user food consumption; score the inputted user activities; track scores over time; and display one or more representations of user progress based on the scores.

For example, an integrated disease management system for enhancing employee health may include: an outer box; a plurality of closed pouches within the outer box; a dietary supplement within each of the plurality of pouches; and a non-transitory computer-readable storage medium storing a set of instructions capable of being executed by a smartphone, that, when executed by the smartphone, causes the smartphone to: receive input of user activities including user consumption of the dietary supplement; and display one or more representations of user progress based on the inputted user activities; and a library of videos directed to health and wellness topics.

In some variations, an integrated disease management system for enhancing employee health may include: an outer box; a plurality of closed pouches within the outer box; a dietary supplement within each of the plurality of pouches, wherein the dietary supplement comprises a pill, tablet, gummy, powder, softgel, chew, capsule, or gel; wherein each pouch comprises a front having a first panel and a back, wherein the first panel includes an educational message; and a non-transitory computer-readable storage medium storing a set of instructions capable of being executed by a smartphone, that, when executed by the smartphone, causes the smartphone to: receive input of user activities including: user consumption of the dietary supplement, user physical activity, and user food consumption; score the inputted user activities; track scores over time; and display one or more representations of user progress based on the scores; and a library of videos directed to health and wellness topics, further wherein the set of instructions causes the smartphone to access the library of videos for display to the user.

Also described herein are methods of improving a user's health by multiple daily messaging encouraging routine healthy behaviors, the method comprising: providing a plurality of pouches, wherein each of the plurality of pouches comprises a dietary supplement, further wherein each pouch comprises an educational message; providing a non-transitory computer-readable storage medium storing a set of instructions capable of being executed by a smartphone, that, when executed by the smartphone, causes the smartphone to: receive input of user activities including user consumption of the dietary supplement and one or more of: user physical activity and user food consumption; score the inputted user activities; track scores over time; display one or more representations of user progress based on the scores; and transmit the received input of user activities or scores to a remote server; receiving, in the remote server, the input of user activities or scores; and tracking, in the remote server, user consumption of the dietary supplement and/or user activity.

Any of these methods may also include delivering customized educational content to the user based on the input of user activities or scores, and/or connecting the user to a medical professional based on the input of user activities or scores.

In any of these methods, the set of instructions may cause the smartphone to transmit the received input of user activities or scores to a remote server using encryption to protect the user's privacy.

Also described herein are methods of improving a user's health by multiple daily messaging encouraging routine healthy behaviors, the method comprising: providing a plurality of pouches, wherein each of the plurality of pouches comprises a dietary supplement; further wherein each pouch comprises an educational message and one or more images corresponding to the educational message; providing a non-transitory computer-readable storage medium storing a set of instructions capable of being executed by a smartphone, that, when executed by the smartphone, causes the smartphone to: receive input of user activities including: user consumption of the dietary supplement, user physical activity, and user food consumption; score the inputted user activities; track scores over time; and display one or more representations of user progress based on the scores; receiving, in a remote server, the input of user activities; tracking, in the remote server, user consumption of the dietary supplement and user activity.

For example, a method of improving a user's health by multiple daily messaging encouraging routine healthy behaviors may include: providing a plurality of pouches, wherein each of the plurality of pouches comprises a dietary supplement, wherein the dietary supplement comprises a pill, tablet, gummy, powder, softgel, chew, capsule, or gel; further wherein each pouch comprises an educational message and hidden text or image related to the educational message that is revealed only when opening the pouch; providing a non-transitory computer-readable storage medium storing a set of instructions capable of being executed by a smartphone, that, when executed by the smartphone, causes the smartphone to: receive input of user activities including user consumption of the dietary supplement and one or more of: user physical activity and user food consumption; score the inputted user activities; score the inputted user activities; and display one or more representations of user progress based on the inputted user activities; receiving, in a remote server, the input of user activities; tracking, in the remote server, user consumption of the dietary supplement and user activity; and delivering customized educational content to the user based on the input of user activities.

A method of improving a user's health by multiple daily messaging encouraging routine healthy behaviors may include: opening a pouch comprising a dietary supplement, further wherein each pouch comprises an educational message and one or more images corresponding to the educational message; inputting, into a smartphone, information about user activity including: user consumption of the dietary supplement, user physical activity, and user food consumption; scoring, in the smartphone, the inputted user activities; tracking, in the smartphone, a plurality of scores for the inputted user activity over time; and displaying, on the smartphone, one or more representations of user progress based on the scores. Any of these methods may also include receiving, in a remote server, the information about user activity. For example, the method may include receiving, in a remote server, the information about user activity; and delivering, from the remote server, customized educational content to the user based on the received information about user activity.

Opening the pouch may include exposing a hidden message, as mentioned above. For example, opening the pouch may include tearing the pouch to expose, on the back a fourth panel between a second and a third panel, wherein the fourth panel includes a hidden text or image related to the educational message and is only visible when the pouch is opened.

Any of these methods may include opening a first chamber of the pouch, wherein each pouch comprises at least two sealed chambers, wherein each chamber includes the dietary supplement. Any of these method may also include transmitting from the smartphone the user's scores to a remote server.

Also described herein are methods of improving a user's health by multiple daily messaging encouraging routine healthy behaviors, the method comprising: opening a pouch comprising a dietary supplement, wherein the dietary supplement comprises a pill, tablet, gummy, powder, softgel, chew, capsule, or gel; further wherein each pouch comprises a front having a first panel and a back, wherein the first panel includes an educational message, and further wherein the back comprises a second panel and a third panel that are adjacent to each other when the pouch is closed, and a fourth panel between the second and third panels that is exposed only when the pouch is opened to reveal hidden text or image related to the educational message; inputting, into a smartphone, information about user activity including: user consumption of the dietary supplement, user physical activity, and user food consumption; using the smartphone to score the inputted user activities and track the scores over time; displaying, on the smartphone, one or more representations of user progress based on the scores; receiving, in a remote server, the information about user activity; and delivering, from the remote server, customized educational content to the user based on the received information about user activity.

### BRIEF DESCRIPTION OF THE DRAWINGS

The novel features of the invention are set forth in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a flowchart with the various components of the integrated system and how they interrelate

FIG. 2 shows an exemplary outer box that contains pouches containing dietary supplements.

FIGS. 3A-3D shows sample pouch messages.

FIG. 4A shows the layout of one variation of pouch messages comprising three panels.

FIG. 4B shows the layout of one variation of pouch messages into four panels.

FIG. 4C shows the layout of one variation of the pouch in which an extra seal is present, creating two compartments.

FIG. 5A shows various ingredient compositions of an exemplary dietary supplement comprising gummies.

FIG. 5B shows various ingredient compositions of an exemplary dietary supplement comprising plant sterols.

FIG. 5C shows various ingredient compositions and ratios of exemplary dietary supplements comprising plant sterols.

FIGS. 6-21 shows sample content from the package insert (brochure).

FIG. 22 shows exemplary screen shots from an app including means of monitoring daily activities and tracking weekly performance.

FIG. 23 show examples of various targets for cholesterol and blood pressure management.

FIG. 24 is a flowchart of how patients may be assessed and provided the system described herein.

FIG. 25 shows how a multi-modal system described herein may be incorporated into corporate health and wellness programs.

### DETAILED DESCRIPTION

Described herein are products, systems and programs that are aimed at the treatment of various health conditions or management of health and wellness and thus the prevention of medical conditions and their complications.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

An Integrated System

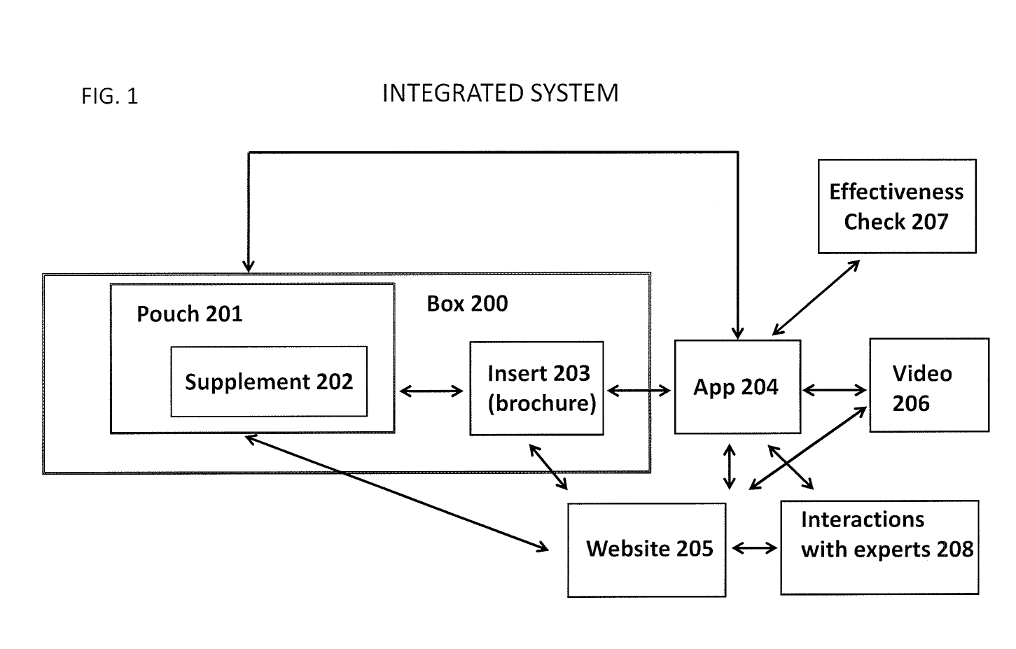

The integrated systems approach described herein comprises previously disparate components, all seamlessly integrated to maximize efficacy in preventing, treating or managing health and wellness conditions such as borderline high or high cholesterol, prehypertension or hypertension and other chronic conditions. FIG. 1 shows the various components and how they work together to provide optimum benefit.

The Box 200 contains multiple pouches 201 which contain one or more dietary supplements 202 within the pouch. The box also contains an insert 203 which may take the form of a multi-page brochure that fits within the box. There is an interactive app 204 that the user can download on a phone, computer or tablet, a website 205 and educational/motivational videos 206 that are all part of the integrated system. There is an effectiveness check 207 with results that can be input into the app 204. The effectiveness check may be performed by an outside laboratory and may comprise a self-assessment completed by the user. There is also interactions with experts 208, wherein these experts are doctors, nutritionists, nurses, coaches, and other healthcare providers that interact with the user in person, via telephone, web, video, chat, or other means.

In some cases, only a subset of these components are present in a system. For example, in some cases, there may be no effectiveness check or video content. In other cases, the component may be merged. For example, the interactive app may take the form of an interactive website that can be accessed and utilized by mobile phone or tablet. In other words, there may be no app, but its functionality will be integrated into a website that may serve additional information as well.

Many of the components of the integrated system interrelate, with the goal of exposing the user repeatedly to the same behavior change messages, until those behavior changes become routine. Put another way, frequent, unexpected and engaging and fun messaging of improved behaviors is likely to drive adherence to behavior change.

In some cases, the goal is to expose the user to the same or similar messages at least twice a day. In other cases, the goal is to exposes the user to at least three, four, five or six or more of the same of similar messages per day or over a longer duration during the week. Importantly, these messages should be delivered through more than one modality if possible. For example, the user could read a message on a pouch in the morning, then receive a push notification later in the day, then re-read a similar message on the pouch in the evening, then watch a video during the evening and then confirm his adherence to one or more behavior change messages in the night. This combination of encouraging behavior change through more than one modalities is called multi-modal messaging and is designed to create healthy routines for the user. The use of multi-model messaging is also to create synergistic benefits that have improved efficacy beyond what might be expected from the components used individually or in combination.

Packaging

In general, packaging is designed to serve one or more purposes including protecting the product (i.e. dietary supplement) during storage and shipping, keeping the dietary supplement fresh and stable (maximizing shelf life), keeping the dietary supplements in quantified units (such as 4-6 pills or gummies per day) to help the user to track his/her intake for the day, providing education about the health condition and how to treat that condition, to entertain or amuse the user. Packaging may comprise one or more of the following components
1) Outer box (or caddie), which may comprise one or more inner dividers designed to separate individual pouches
2) Unit dose pouches or packs for daily, multi-daily (ie more than one day), or weekly use. In some cases, one pack may be used every 4, 6, or 8 hours as an example, or in other words in one day the user may consume 1, 2, 3, 4, etc. packs.
3) One or more package inserts (such as multipage brochures or single page inserts)
4) External packaging (shipper boxes that are received by the end user at their home for example)
5) A bottle (made of plastic, glass, metal or the like) which itself may be placed within an outer box.

A preferred embodiment comprises the outer box, a supply of 30 pouches within the box, a package insert with behavior change messages and a shipper box. These components are described in more detail below:

Outer Box

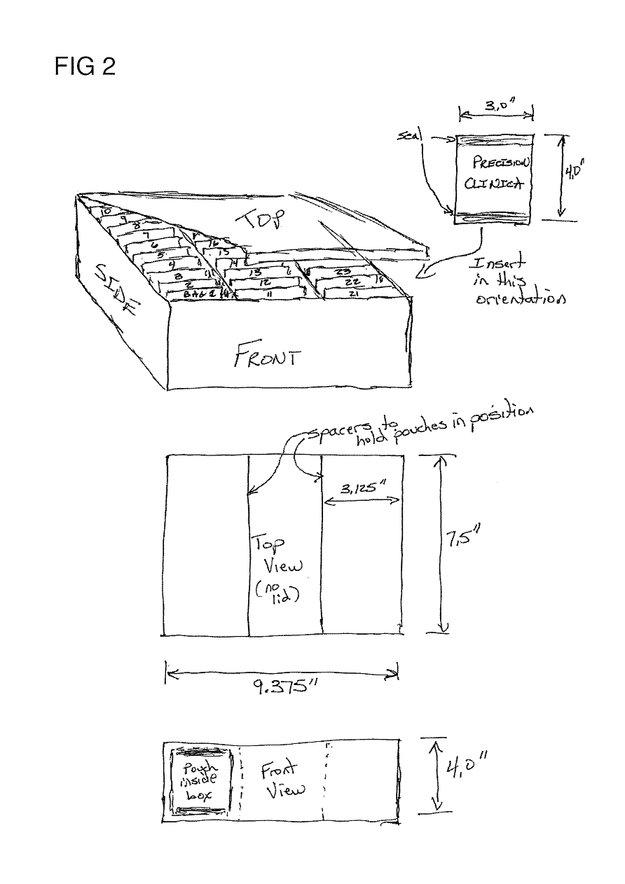

The outer box (or caddie) is designed to carry a specified quantity of pouches. For example, FIG. 2 shows a representative box holding multiple pouches arranged in rows. In this case, 30 pouches are packaged in 3 rows although different pouch quantities and numbers of row (1 row, 2 rows, 3 rows, 4 rows, 5 rows, etc) are possible. The outer box may have an internal divider or spacer. The box is generally made of paper or cardboard, although other materials are possible such as plastic or metal. The box is sized based on the sizes of the pouches and vice verse, to ensure that the pouches do not slide, fall or move during shipping and storage. Placement of the pouches into the box may be automated, semi-automated or may be placed by hand.

The outer box may have a dispensing mechanism which comprises a cutout from a portion of the box which allows the user to use a finger to remove a pouch. The cutout may be linear and/or symmetric, and in other cases, may have a curved edge that prevents pouches from falling out.

The box itself contains text and images, including nutritional, ingredient and other information. In some cases, the box may itself have behavior change information. In other cases, the box may provide a link to the website, and the website may provide additional information including verification of quality, identity, toxicology testing and the like.

Pouches/Packs

Packaging may be chosen to maximize shelf life and to provide convenience to the user. This may involve a daily use or single use pack that has one or more layers including but not limited to a paper layer, plastic layer or metal or foil layer, or a combination thereof. The inner layer (if there are multiple layers) is chosen to prevent degradation of the dietary supplement, while preventing evaporative loss or other degradation that could reduce efficacy or otherwise affect taste, texture or color. An outer layer may also be added to protect the printing or change the appearance, e.g. create the matte look. The outer layer will have printing, printing which can serve to educate the user about the product, the medical condition or to promote behavior change that may help or aid in the treatment or prevention of a medical condition. Pouches can be produced such that they require the user to tear the pouch to open or could be resealable. A resealable pouch may help keep the dietary supplements fresh throughout the day and enable the user to open and close the pouch to access the supplements throughout the day.

In one embodiment, the package contains a daily supply of dietary supplement, and each time the user picks up and/or handles the package, he or is she provided an important message about lifestyle modification that may be additive or synergistic relative to the effect of the dietary supplement on that medical condition. In some cases, the messages on the package will reference, be similar to or be substantially the same as information, guidelines or guidance provided by one or medical societies or well respected organizations. In this way, the user will learn about important lifestyle changes that have been scientifically proven to improve health. For example, in the case of cholesterol maintenance, the messages may be adapted from or based on guidance from the NCEP (National Cholesterol Education Program), the NIH, American Heart Association (AHA), American College of Cardiology (ACC) or similar medical or patient societies from Europe, Asia, South America, North America, or any of their member countries.

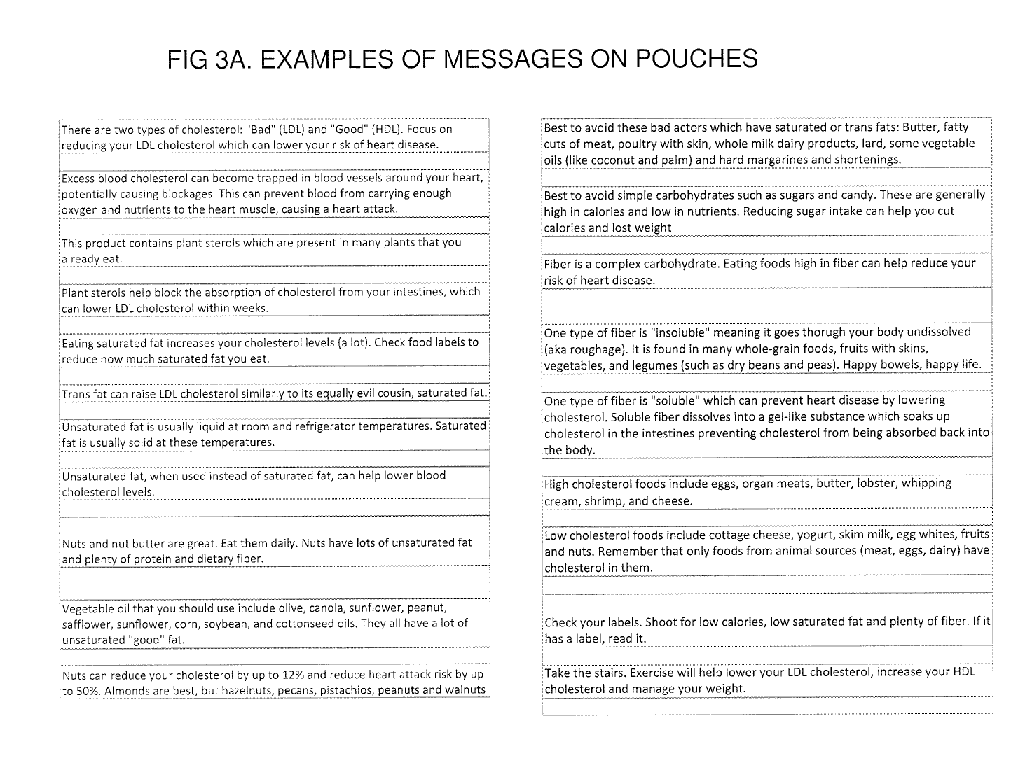

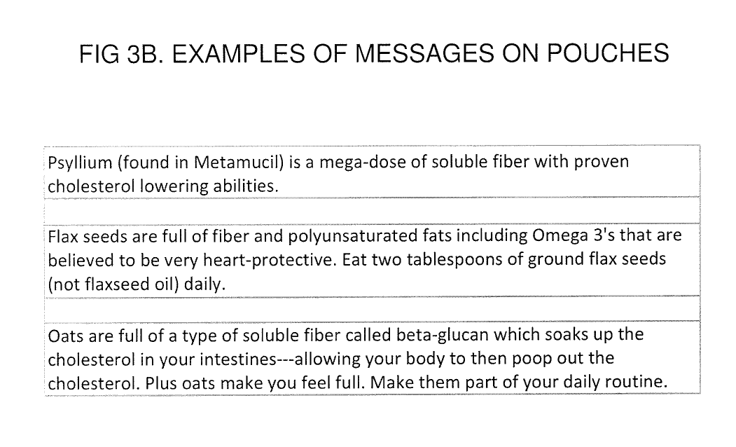

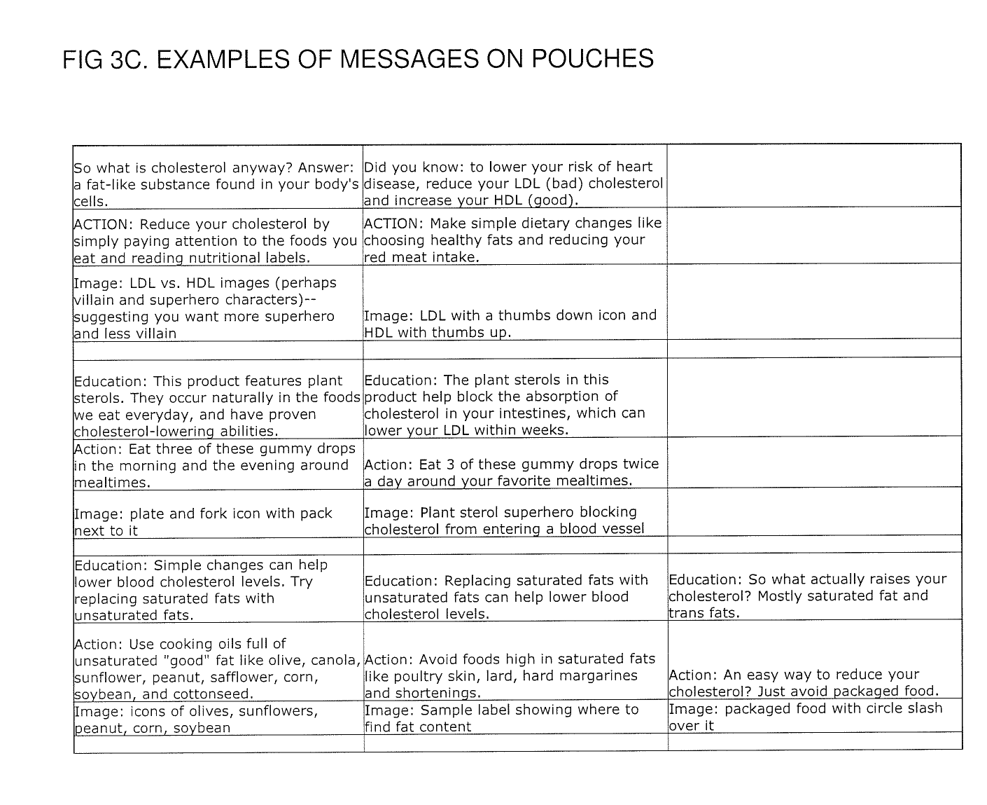

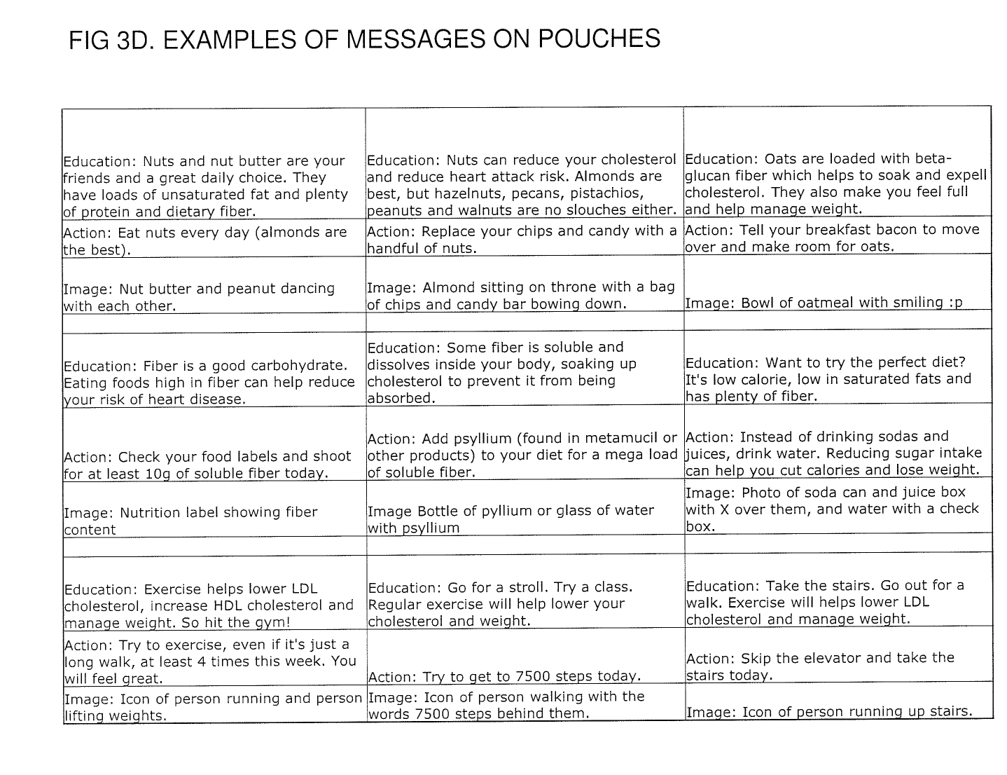

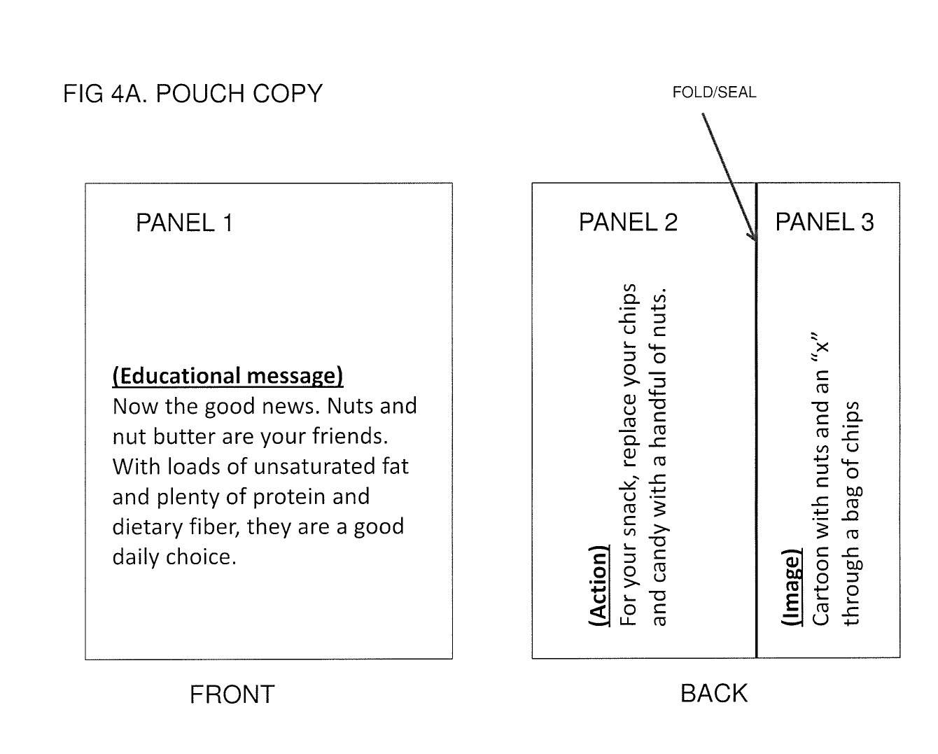

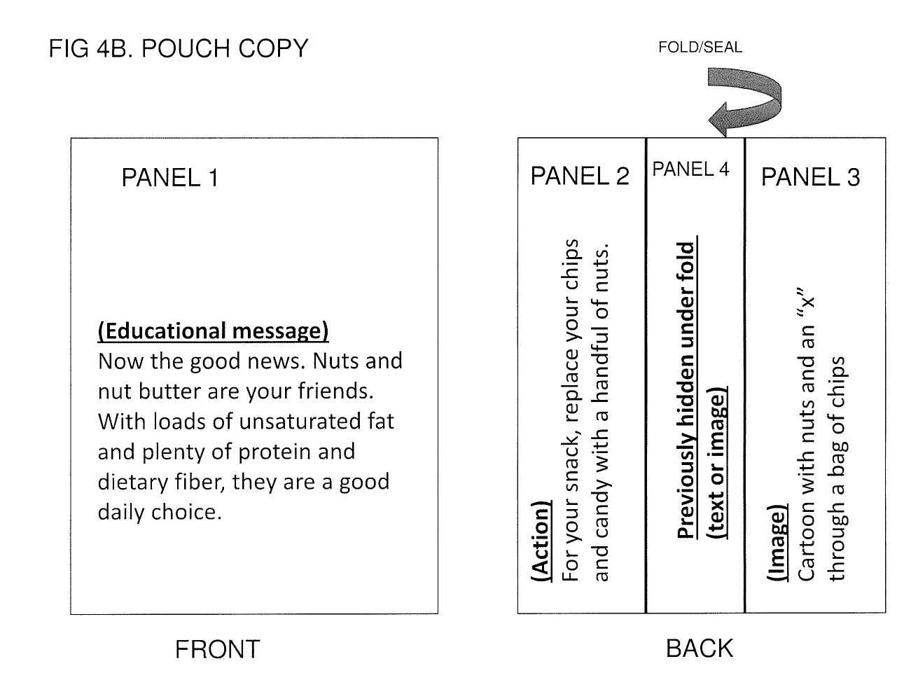

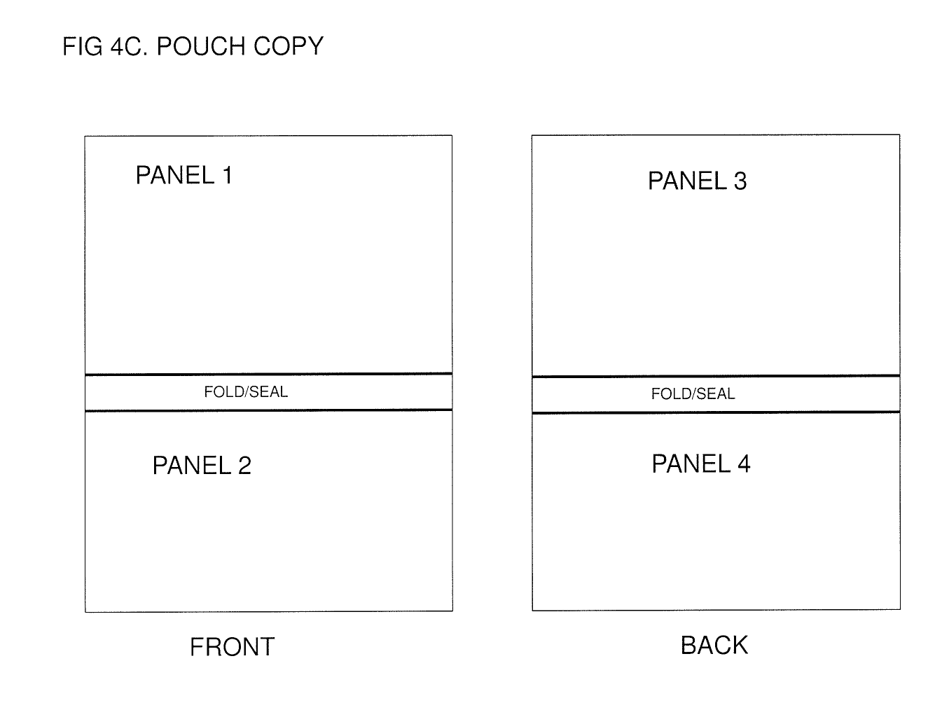

Example of these messages can be found in FIGS. 3A-D. This list is meant to be representative and not limiting. The format of the behavior change messaging may take many forms. In some embodiments, the messaging will be broken into two, three or four or more components on the daily dose packaging as can be found in FIG. 4A or 4B. This may comprise:

1) Basic information about a condition or a practice (For example: Replace saturated fats with unsaturated fat. This simple change can help lower blood cholesterol levels.)
2) An intervention that the user can do right away (For example: Use cooking oils full of unsaturated “good” fat include olive, canola, sunflower, peanut, safflower, corn, soybean, and cottonseed instead of butter or lard)
3) An image that reinforces the points above and may be engage or amuse the user (For example: An image of bottles of oil with a “check” and an image of lard with an “X”)
4) A “reveal” opportunity involving allowing the user to unfold or look under the fold to find additional content such as images, text or both. This reveal opportunity is designed to engage and delight the user.

It is important to provide repetitive messages to the users. In some cases, there will be 6 or 7 different messages on the pouches for a given size box (for example a box of 30 pouches). In other cases, there will be 12 or 14 or more messages per box. In flexographic printing, the number of different messages per box is a function of the length of the pouch and the circumference of the printing cylinder that prints the pouches. Digital printing of the pouch may also be used in order to increase the number of different messages available on pouches in the box.

In some cases, the pouch may have an additional seal mid-way or substantially mid-way down the package as seen in FIG. 4C. This additional seal would enable each daily pack to have two sealed components, for example containing 3 gummies for a morning dose and 3 gummies for an afternoon dose.

In some embodiments, the pouch may be designed to facilitate scanning by a mobile phone or similar instrument. This could involve scanning a QR code for example to help track which pouch has been consumed and/or help determine patient adherence to taking the dietary supplement. In other cases, the user may take a photo of the opened package and the phone, app or other program will confirm by the package shape, tear in the pouch or other feature that the user has consumed the supplement. In other cases, each pouch will have a unique or substantially unique identifier (in addition to other printed features such as a lot number). A photo or scan of the unique identifier will serve to confirm user adherence.

Dietary Supplements

Dietary supplements refers to any compounds that provide improved health benefit to the user that take the form of pills, tablets, gummies, powders, softgels, chews, capsules, gels, and other form factors. In some cases, the dietary supplement may be a prescription product or a doctor recommended or prescribed pharmaceutical.

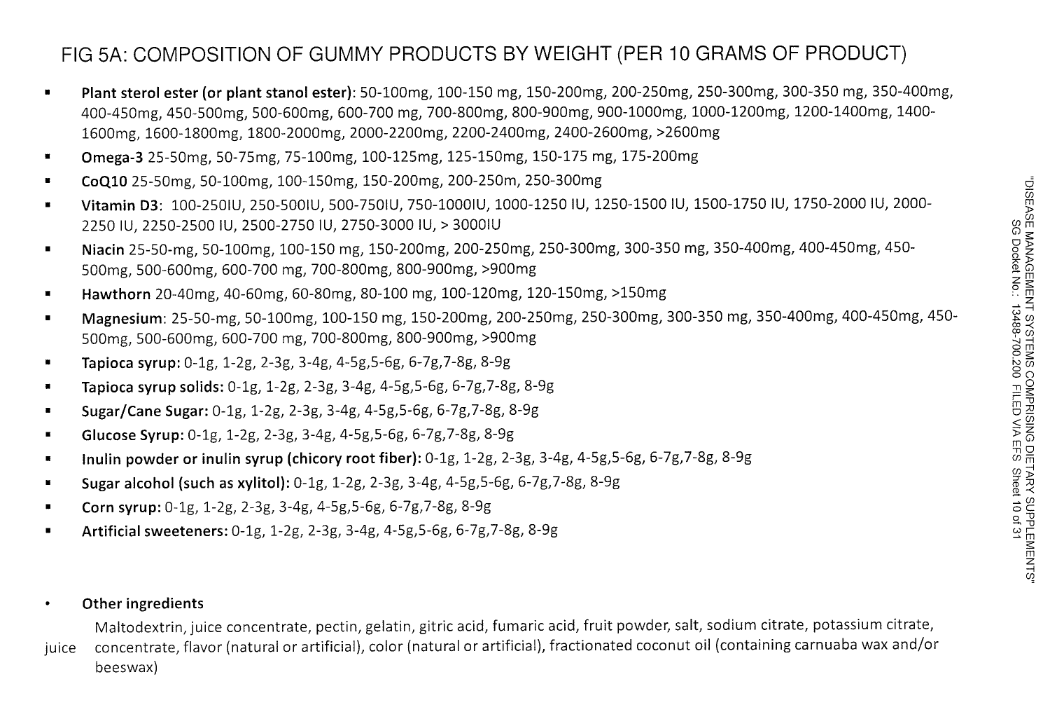

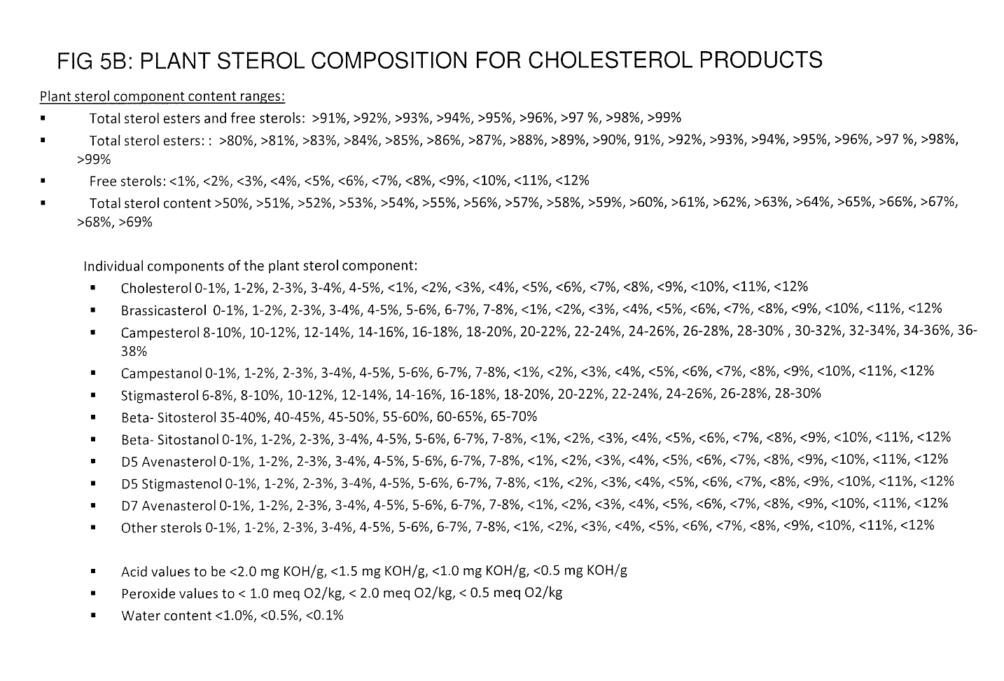

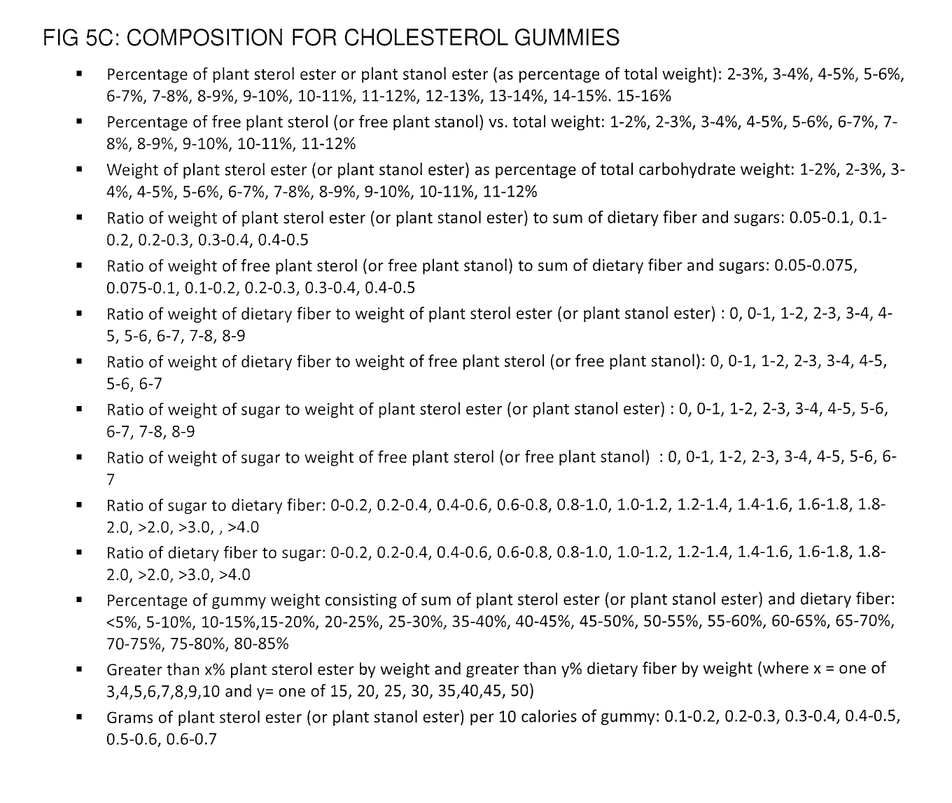

A preferred embodiment is that of a gummy. The gummy may have a variety of ingredients as described more in FIGS. 5A-C.

In some embodiments, the dietary supplement contains a plant sterol or plant sterol ester (or plant stanol/plant stanol ester) and is intended to treat or manage high cholesterol, benign prostatic hyperplasia (BPH) or any other previously mentioned condition. The composition of the plant sterol ester (and underlying free plant sterol) is of utmost importance. The plant sterol itself comprises various types of phytosterols as shown in FIG. 5B. This plant sterol component must have the right composition to maximize clinical efficacy while maintaining taste, color and manufacturability.

Package Inserts

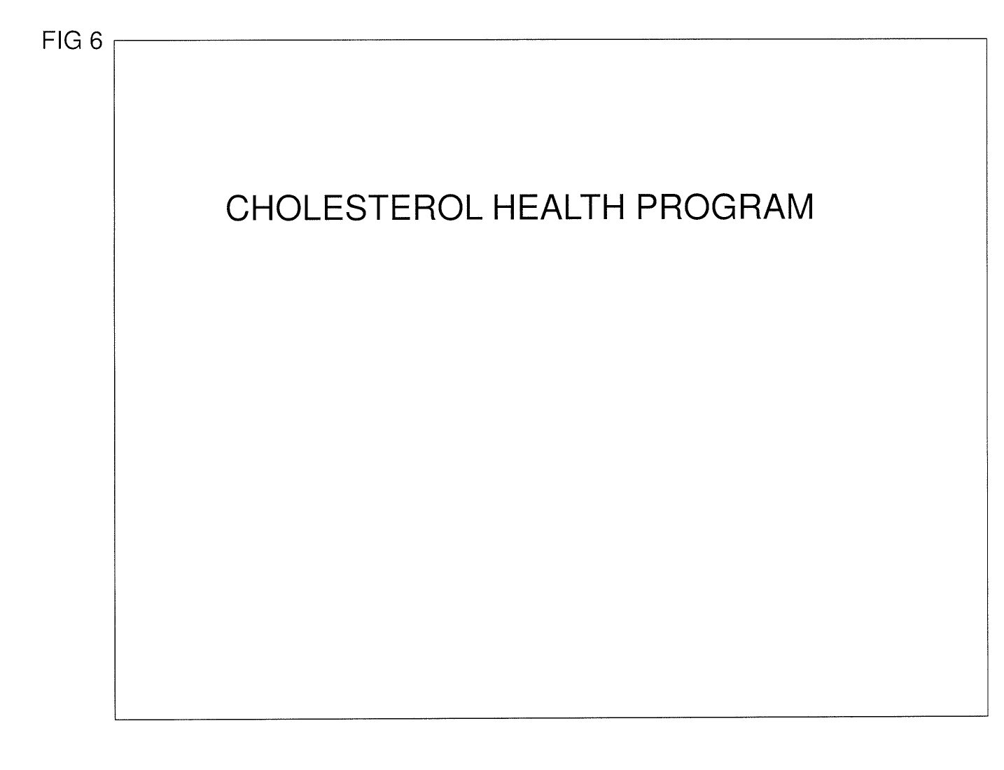

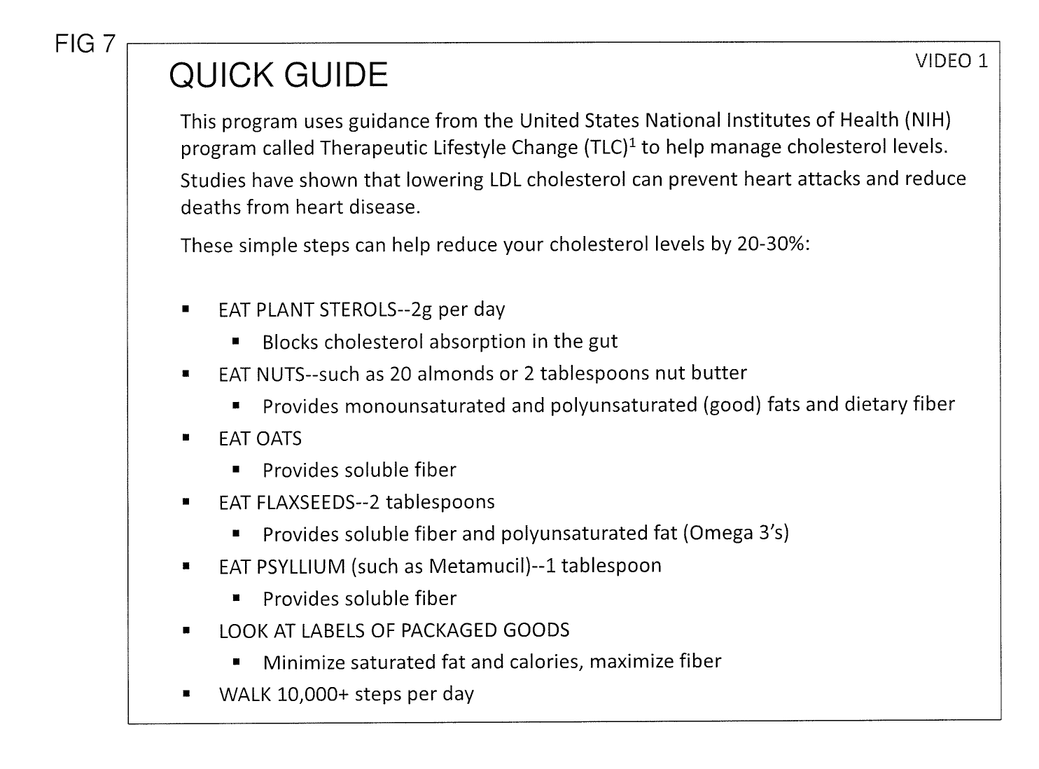

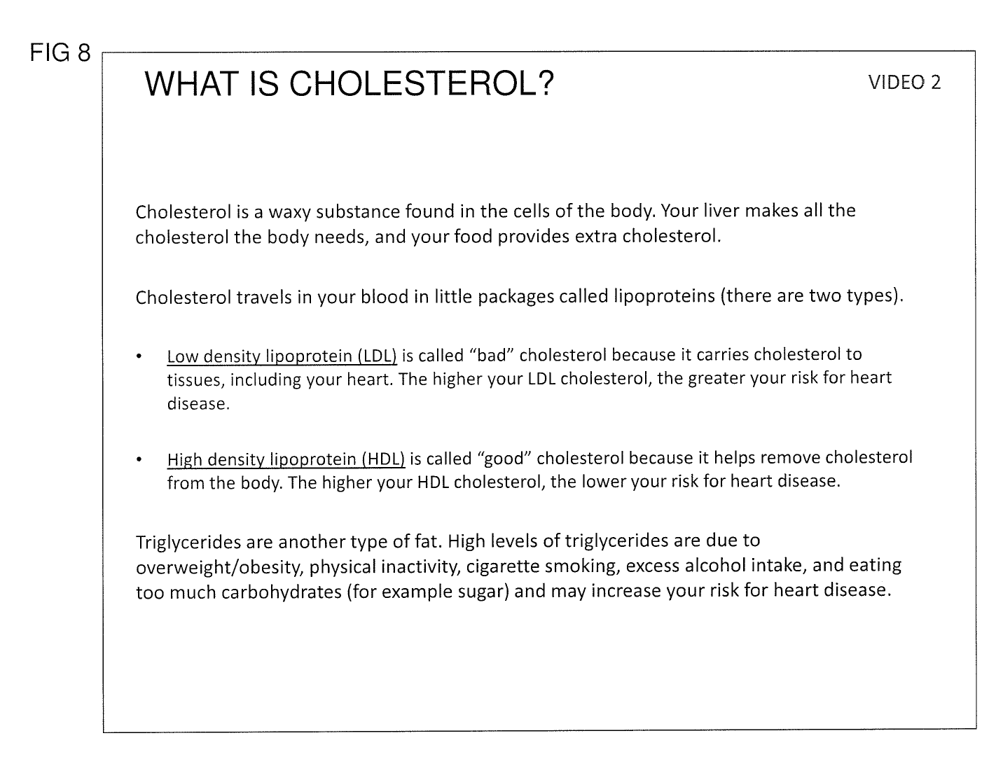

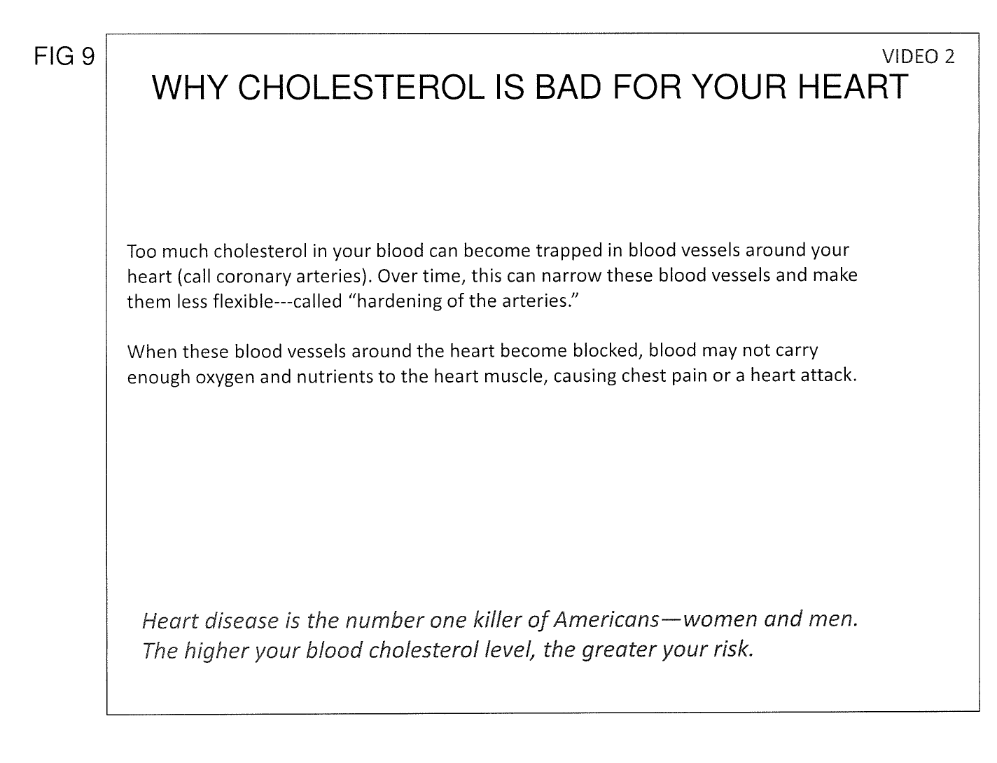

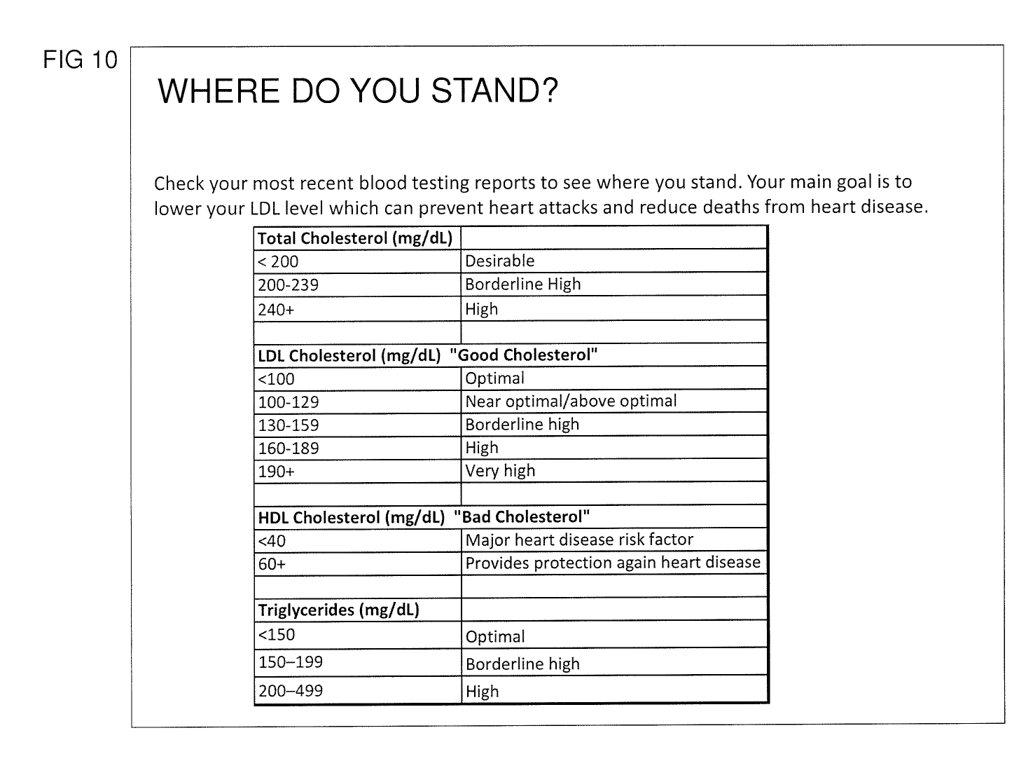

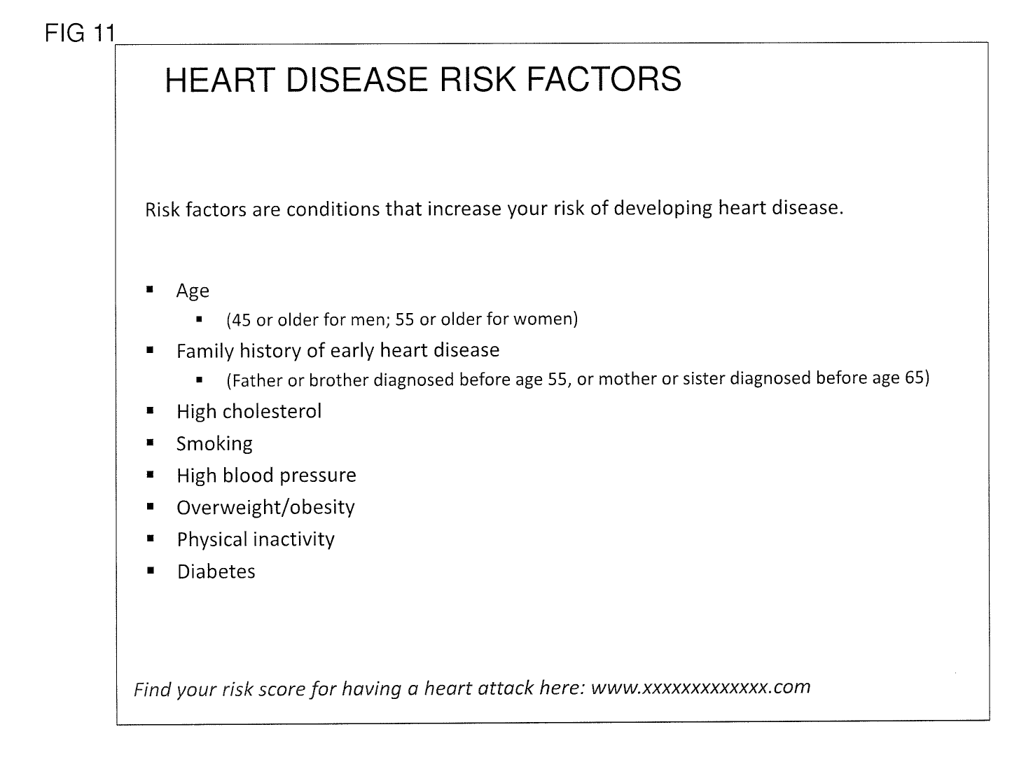

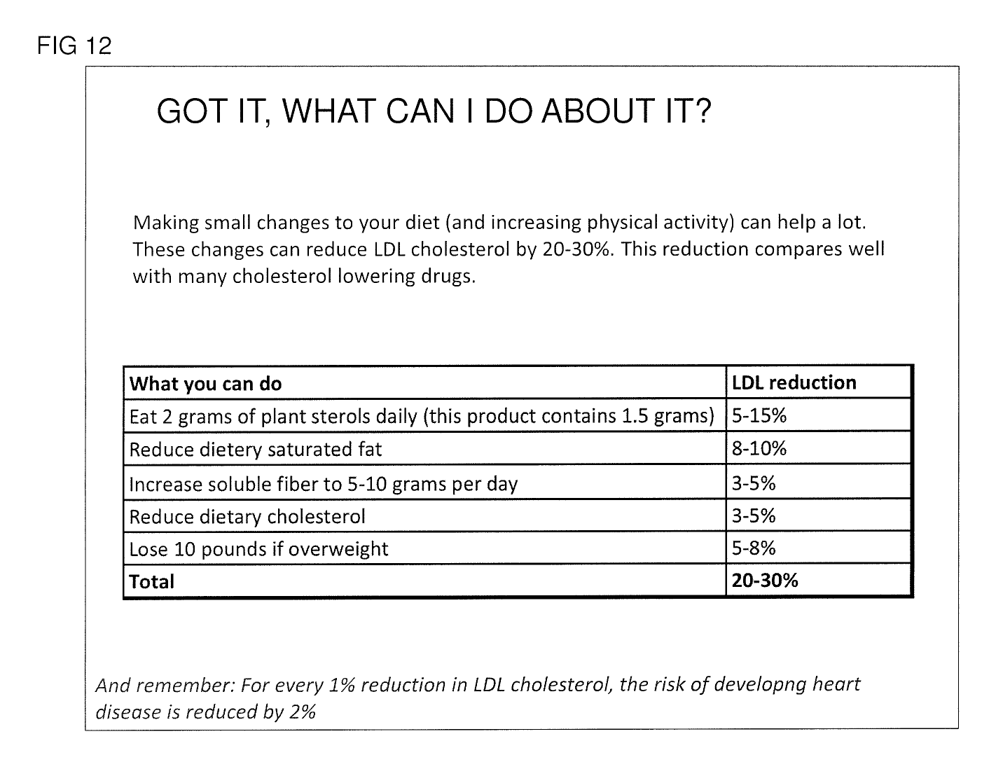

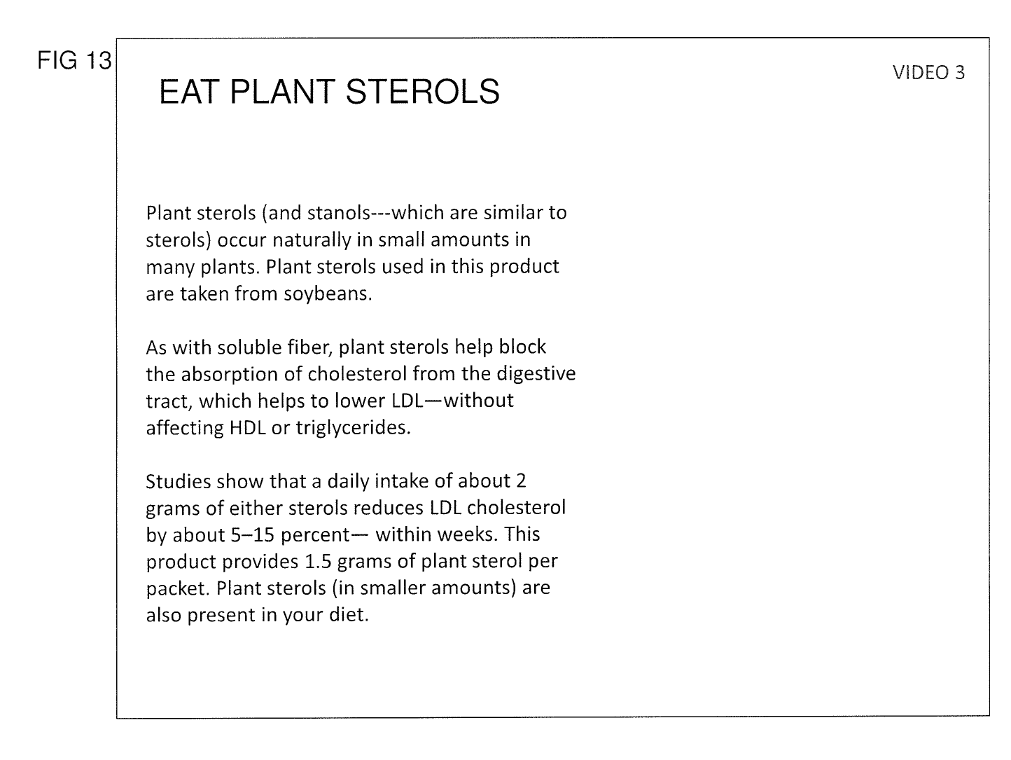

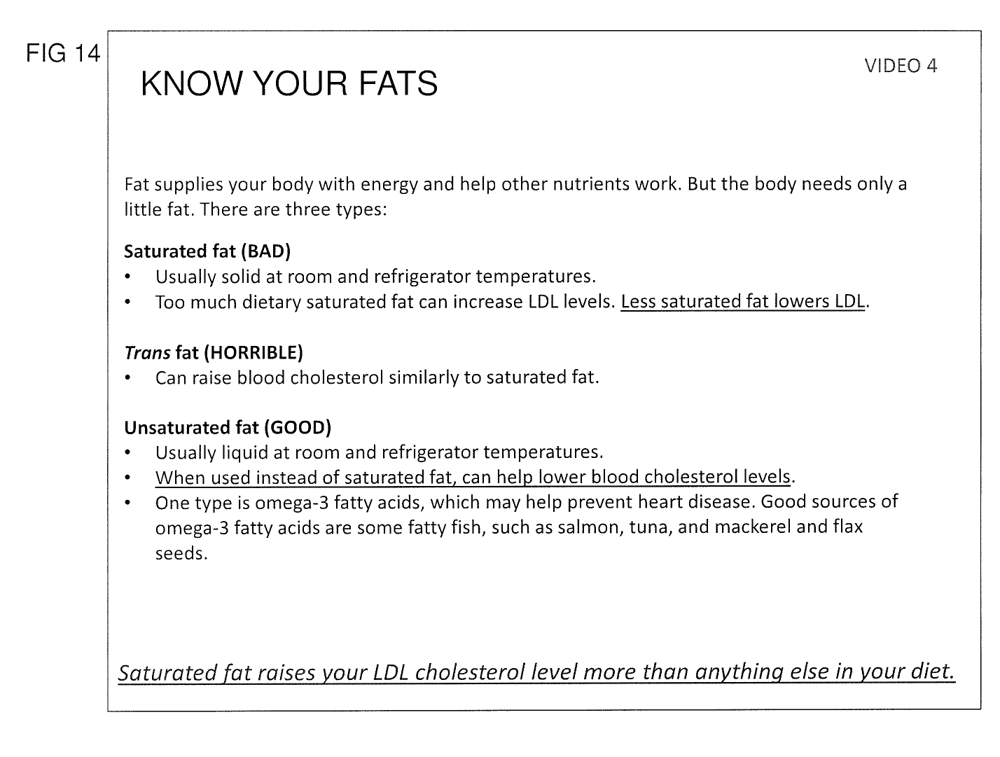

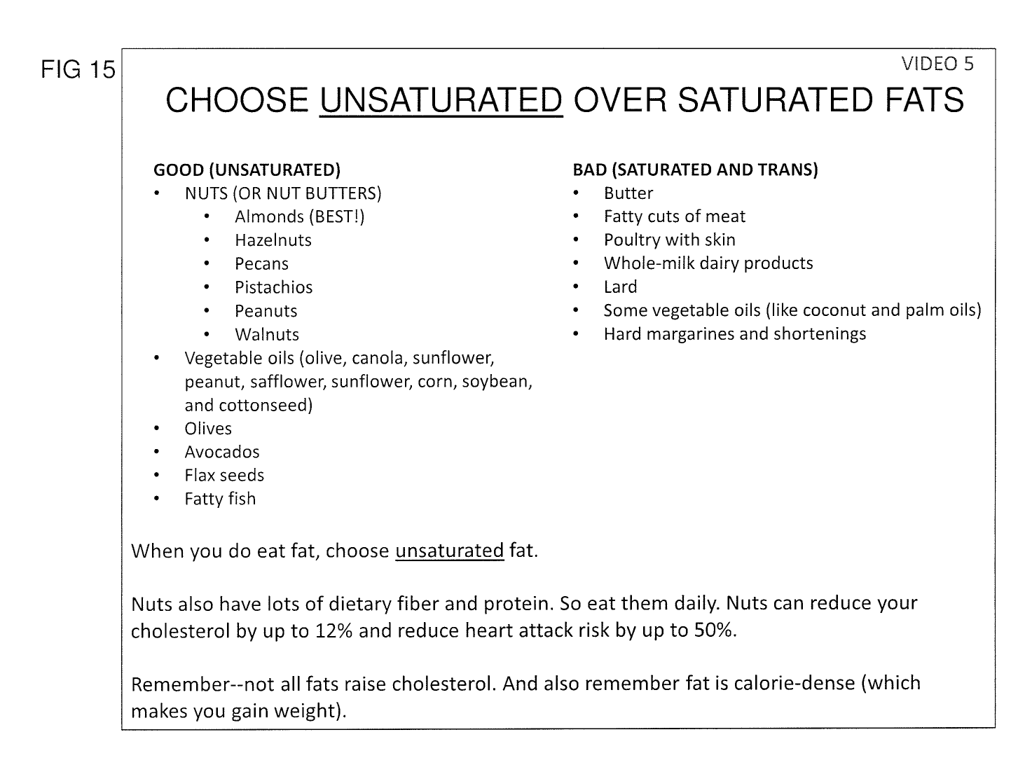

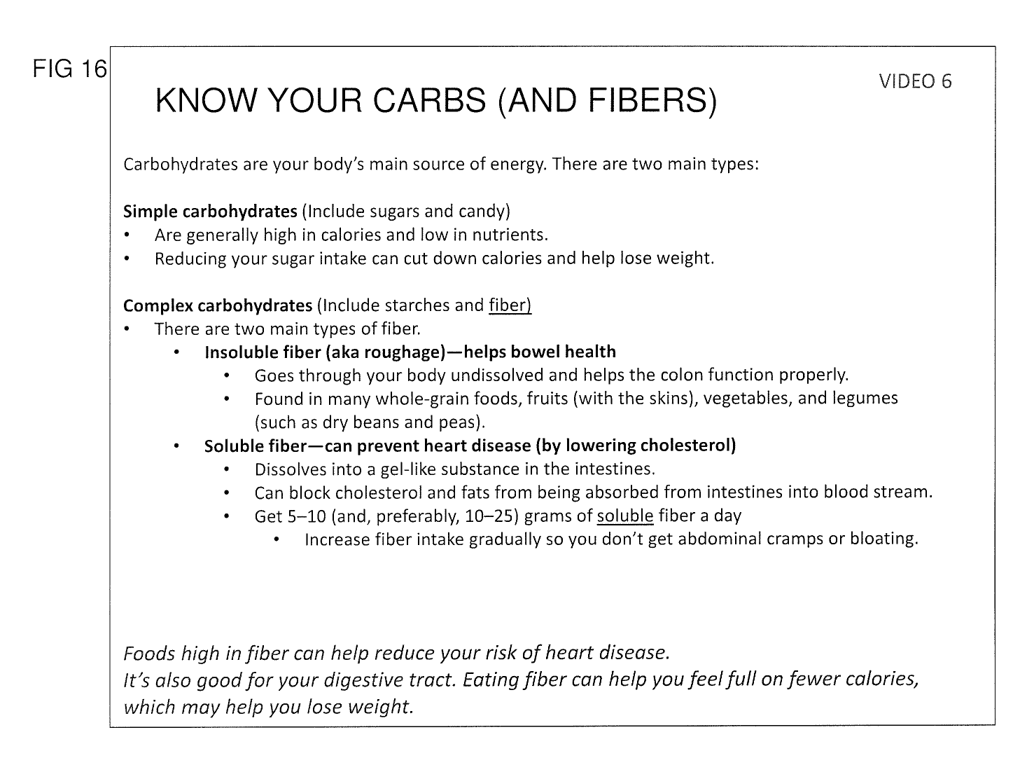

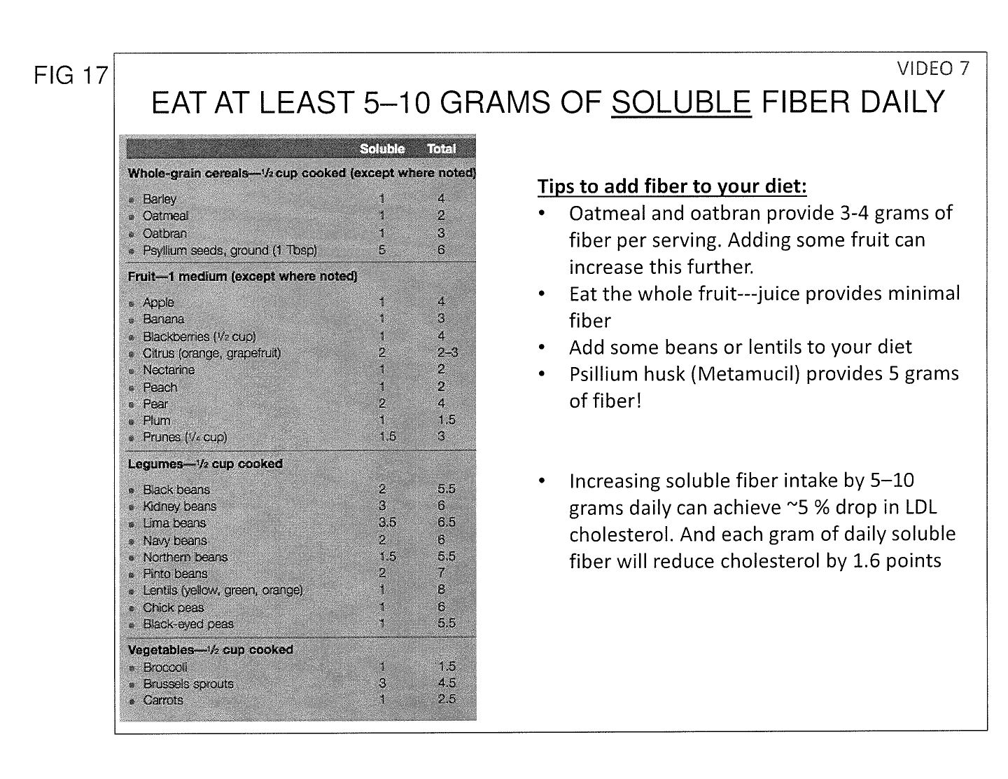

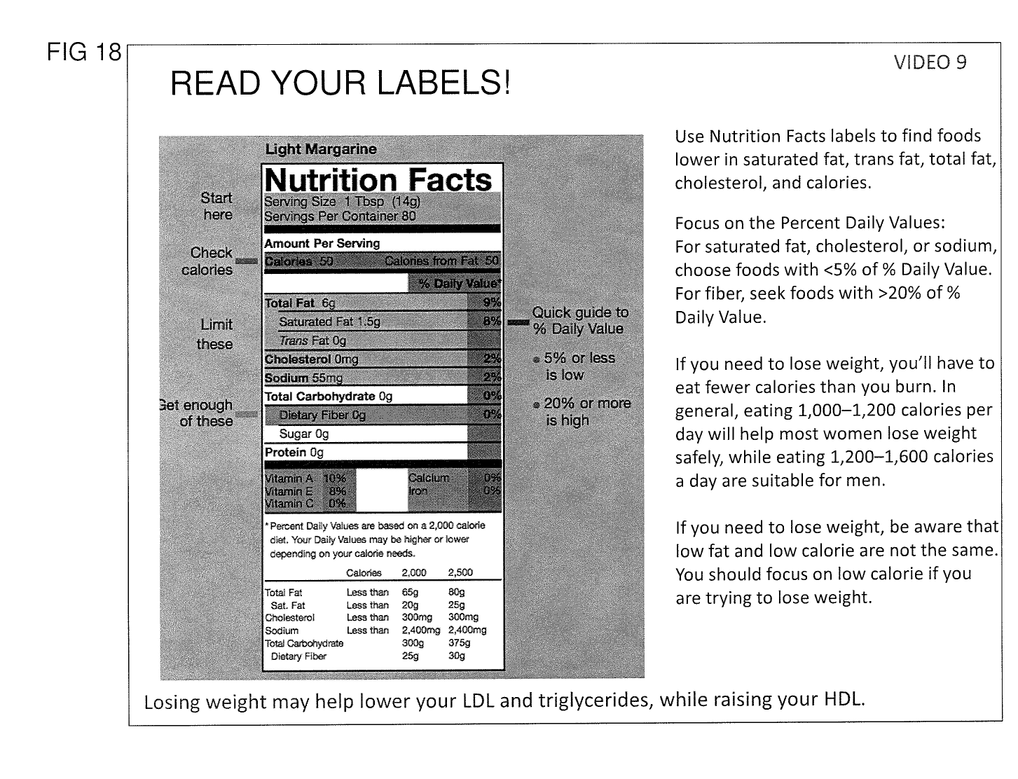

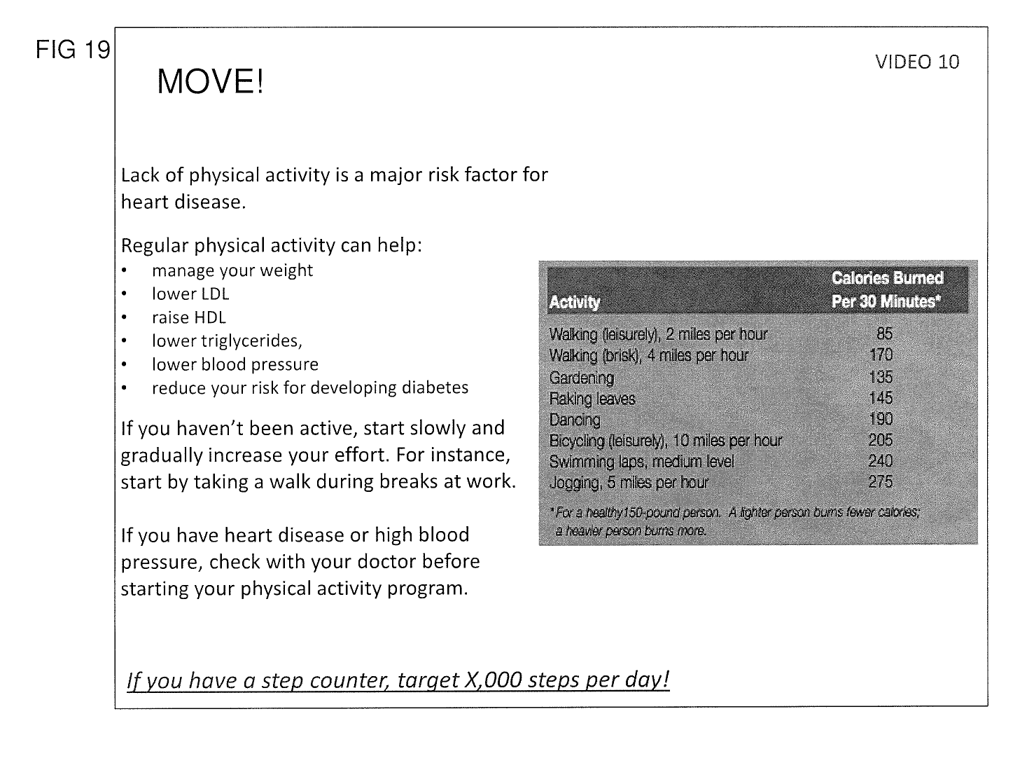

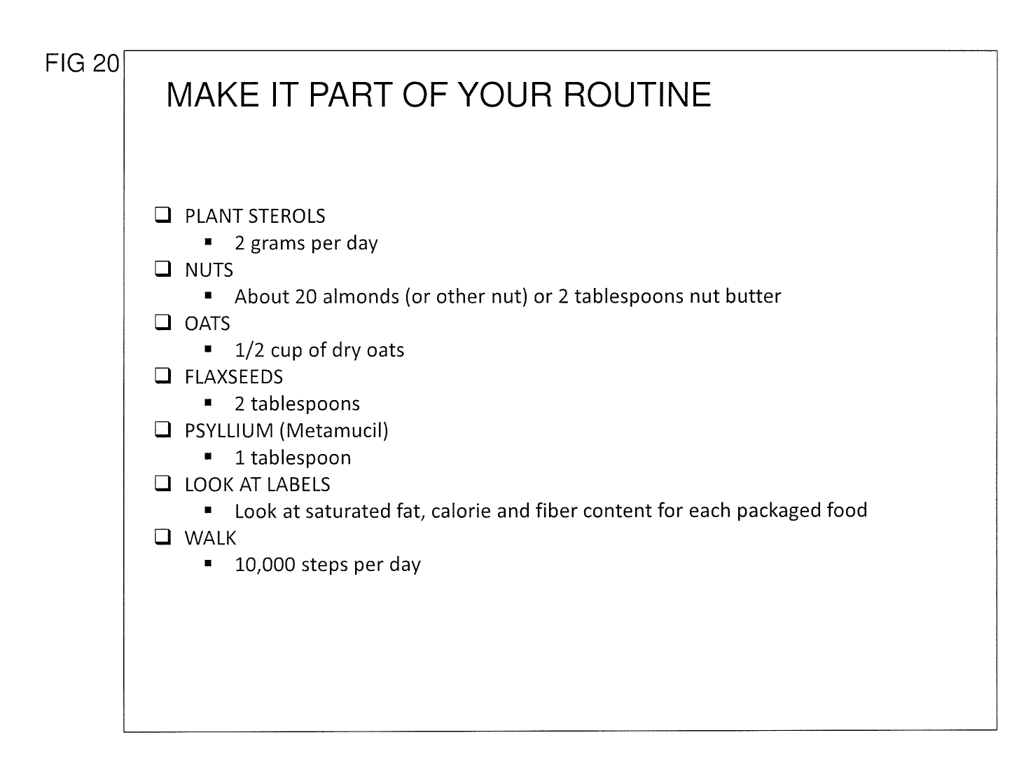

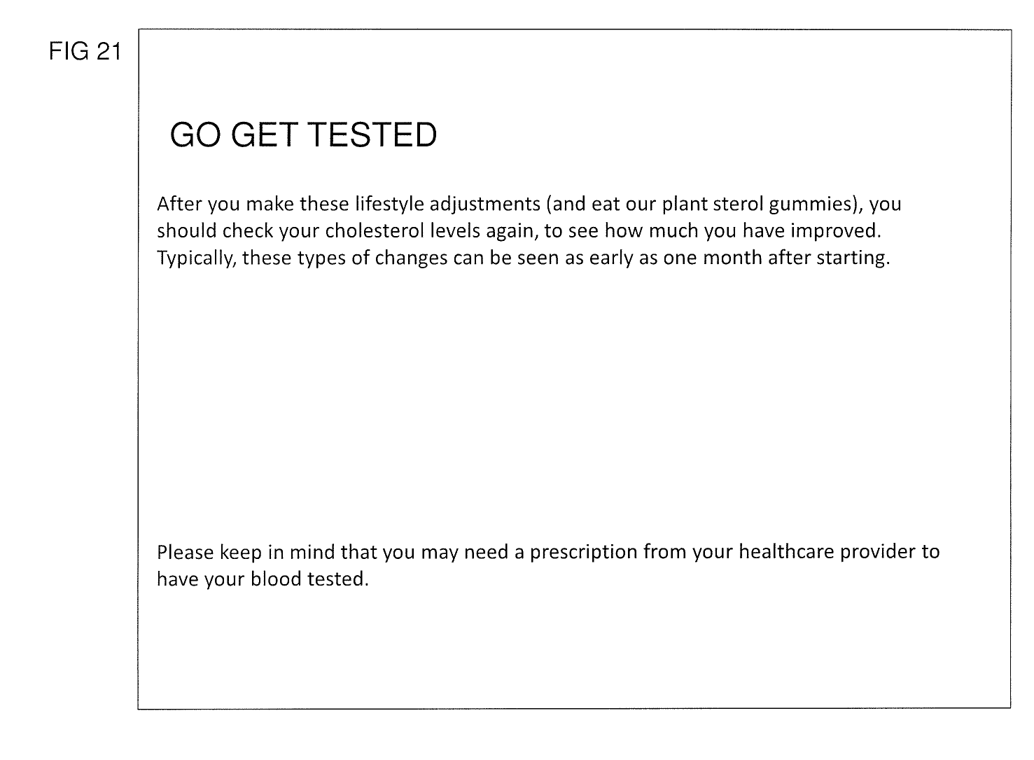

Package inserts may include standard marketing information to promote subsequent ordering of the product via web or telephone. In other cases, a multi-page brochure will be provided. Representative content of this multipage brochure can be found in FIGS. 6-21. In general, content for the brochure and other inserts will be taken from one or more guideline documents from various medical societies or patient organizations and modified to be more understandable and practicable by the end user. Content from the package inserts (brochure) will be similar to content found on the individual pouches as it is beneficial for the user to see the behavior change messages repeated using multiple formats. Thus, similar messages will also be provided by the interactive app and video content albeit via another modality of communication.

Interactive App

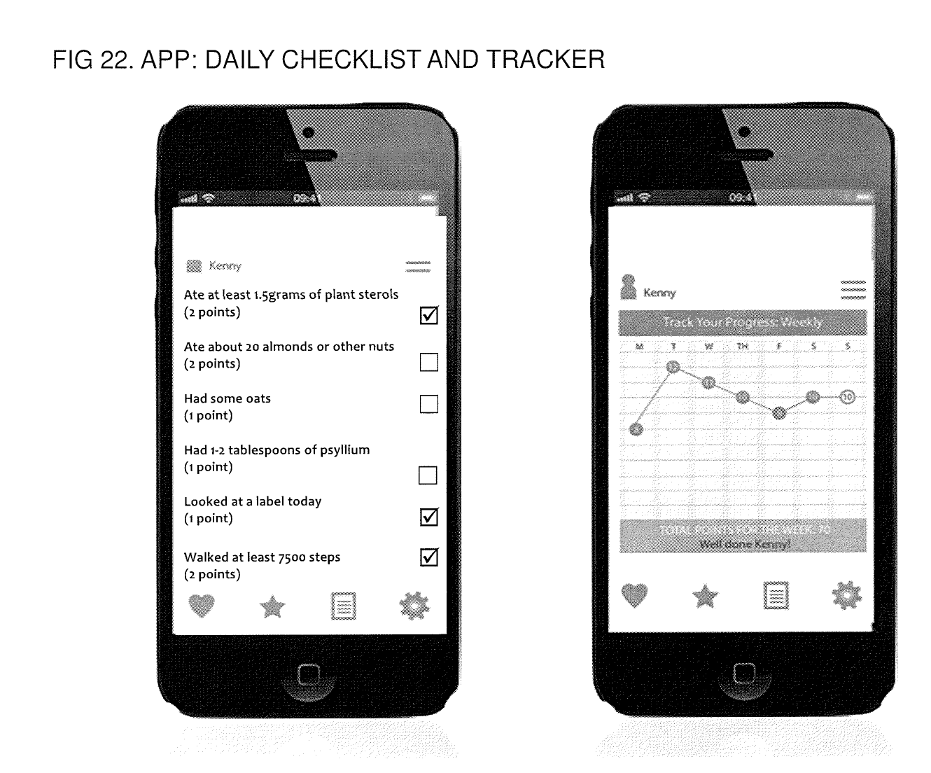

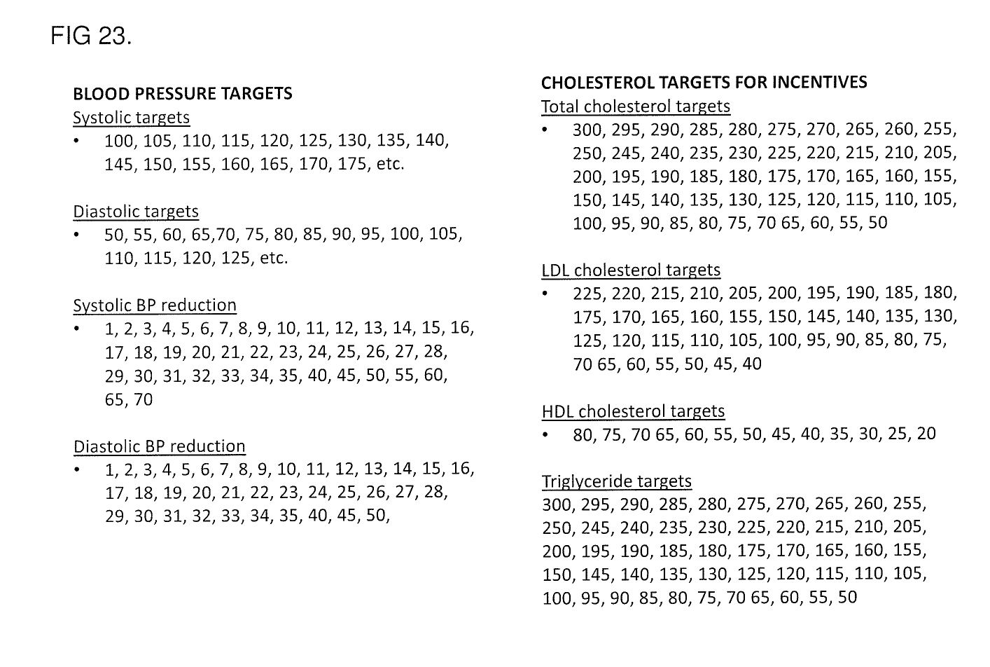

The system also optionally includes an interactive app (shown in FIG. 22) that also promotes user engagement, drives adherence to the behavior change principles and helps the user form a routine of improved health behaviors. The app may be downloadable by the end user and is compatible with the iPhone OS, Android, Windows and other operating systems. Further, data from the app (or from a website that can be accessed and/or is optimized for use on mobile phones an tables) may integrate into various corporate health and wellness programs (and their related health and wellness tracking software), programs which may be able to reward users who adhere to the behavior change promoted by the app. These programs include by are not limited to companies like Jiff, Redbrick Health, and Limeade. In other words, records of progress or adherence to the behavior change messaging or actual lab results (blood testing results) may be shared with these corporate wellness programs, the corporate wellness aggregators, or the companies themselves to enable tracking of progress and rewards if the user achieves certain point targets for example. In some cases, users may be incented by to achieve various physiologic targets or parameters such as a blood pressure of 140/90 or 120/80 for example or a reduction of 5 or 10 blood pressure points for example. Various targets for cholesterol and blood pressure maintenance and treatment are included in FIG. 23. In some cases, mere participation in the program or a certain number of entries in the app or website over a certain duration of time may qualify the user for a reward or partial reward.

In some embodiments that aim to help manage cholesterol of users, the app will have a daily tracker that will enable the user to enter, track and monitor his/her adherence to the various behavior change activities and principles. For example, in the case of cholesterol management, the user may receive a daily notification during the same time each day (for example 8 pm, 9 pm, etc.) that will ask him to indicate or check the boxes for the interventions he completed that day. In the case of cholesterol management, this may include but not be limited to the following interventions:

Consuming plant sterols (at least 800 mg, at least 1.5 g, at least 2.0 g for the day in divided doses)

Consuming nuts (about 20 almonds or other nuts or 2 tablespoons nut butter)

Consuming oats (at least ⅓ cup or ½ cup per day)

Consuming flaxseeds (at least 1-2 tablespoons per day)

Consuming psyllium (at least 1-2 tablespoons per day)

Looking at labels (to review saturated fat, calorie and fiber content for each packaged food)

Walking a certain number of steps (such as >5000, 7500, 10,000, etc)

In the case of blood pressure management, the user may be asked to perform and track the following interventions:

Increasing magnesium intake

Reducing salt intake

Losing weight (for example 5-10 pounds) or reducing caloric intake

Increasing calcium intake

Increasing soy intake

Consuming CoQ₁₀ and Vitamin D₃

Increasing exercise frequency/walking a certain number of steps

Increasing potassium intake

The app provides a ranking or certain number of points for each intervention and the points may be summed over the day, month, year, or other duration. The app also compiles and retains this information and is able to provide a graphic depiction of how the user is adhering to the program over days, a week, several weeks, month, many months, year, etc. If the user achieves a certain number of points over a certain duration, the user may receive a reward or prize.

The app may also enable the user to enter other user data that is related or unrelated to the health condition he/she is managing. For example, blood pressure (systolic and/or diastolic) may be entered manually or automatically via a blood pressure cuff. Or cholesterol levels may be entered manually or automatically.

In some cases, there is an opportunity to include a measure of outcome (e.g. cholesterol measurement) to the system more explicitly. For example, baseline cholesterol could be determined and entered into the app. Then periodically, cholesterol could be measured and entered into the app. The results/progress could be shared with the employer or corporate health provider and be a basis for rewards but also could be used to provide comments (encouragement or additional education) based on their progress or lack of progress.

In some cases, the interactive app may provide immediate feedback to the user (positive or negative) depending on how well the user is adhering to the behavior change program as well as improvements in various parameters (such as blood pressure).

In other cases, the patient enters baseline measurement (e.g. LDL level) in the app and then periodically gets cholesterol testing and feeds results into the app. The app adjusts recommendations/actions based on the results.

In some cases, the app or website collects baseline information on related measurements (e.g. BMI, typical diet, exercise patterns) and creates customized videos and educational content which is then provided or pushed to the patient. These same measures could be repeated periodically (update BMI, diet and exercise) and the app then reacts or optimizes recommendations based on this newer data.

As mentioned previously, data from the app may flow or sent to into various other programs that track similar data for many users, such as programs and software used by large companies in their health and wellness programs. For example, corporate health programs may provide incentives in the form of gift cards, cash or health insurance premium reduction if the user can achieve a certain number of points over a weekly, monthly or yearly period or if the user enters his data via the tracker at desired or prespecified intervals.

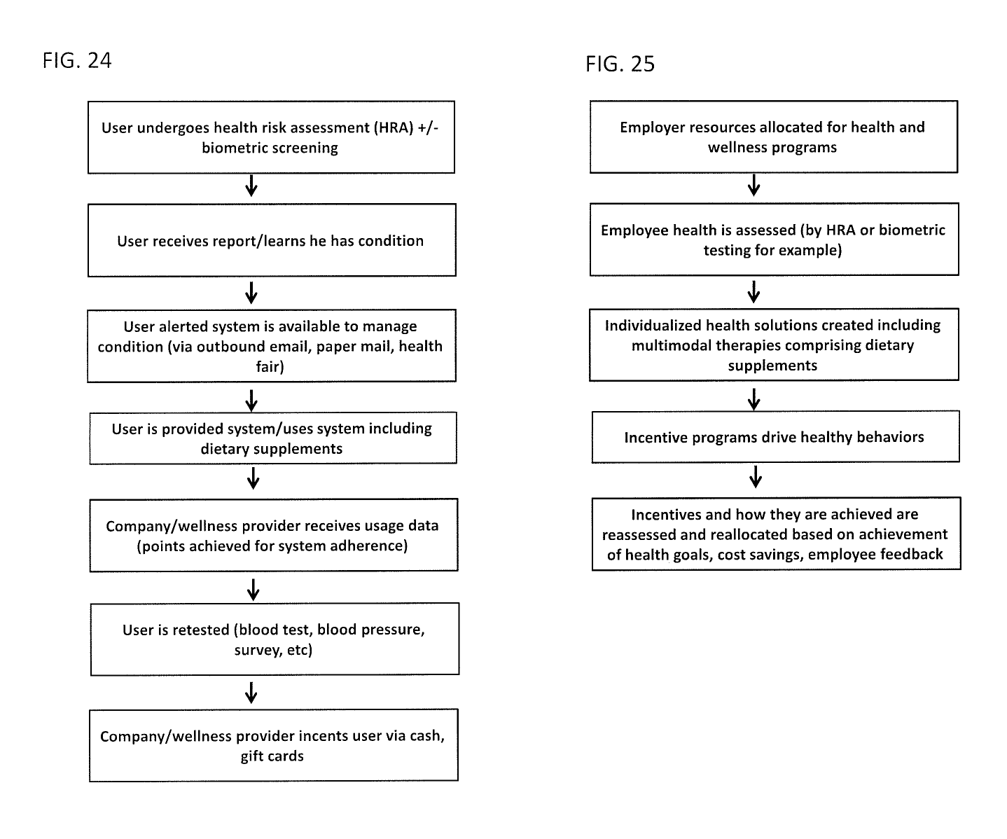

In some embodiments, the app may be customized. The user can enter baseline information into the app (e.g. BMI, exercise habits, normal diet, cholesterol level, blood pressure) which determines which videos or education materials that are recommended or provided. For example if the user indicates he is eating lots of fiber but are not exercising and has a BMI of 35, the app could provide educational info on the importance of exercise and make recommendations for more exercise. For example, the app may suggest various videos of other content designed to promote exercise. Or the app could offer additional points to the activity that is currently being neglected. The app could then track adherence to these activities to see if the user was following the recommendations. Ongoing education, recommendations, rewards, etc. could be based on user compliance. Thus, a customized set of recommendations and an outcomes based feedback loop is built into the system, all controlled by an app, the website and/or its respective algorithms. These algorithms could be optimized by the corporate health aggregator or the corporate wellness program itself to optimize outcomes based on its needs. Examples of these algorithms are shown in FIG. 24. In some cases, the app may not be an app, but in fact be a well designed website customized for mobile browsers or computers.

Video Content

Video content is also part of the integrated system and comprises video segments of various lengths that educate the user about their condition, and provide useful and implementable tips to manage their condition. For example, the videos may be approximately 30 seconds to 10 minutes in lengths and cover a variety of topics. In the area of cholesterol management, topics for the videos may include explanations of what cholesterol is (and its types, namely LDL and HDL), types of fats, benefits of eating fiber, benefits of eating plant sterols, etc. Content will be similar to that from the brochure (FIGS. 6-21) and the app. The video content may feature a white or black screen on which images are drawn as the video is played, so the user can watch images being drawn. This feature is designed to capture the user's attention and make the experience more interesting and interactive.

In some embodiments, the video may feature various characters who are followed from a state of poorer health to a state of improved health. The characters may be depicted in cartoon form and there may be narration of the situation and progress. In many cases, the character will be presented with news of a negative health condition (such as borderline high cholesterol) and the user will be able to see his or her progress in achieving various behavior changes and their effect on the negative health condition. In some cases, there will be a physician (also in cartoon form) who provides guidance to the character (and the viewer) about the condition and various behavior change options. A real doctor or healthcare provider may be part of these videos at the introduction, at the end or points in between.

In some cases, the integrated system will also include access to healthcare providers such as nurses and physicians who can provide additional counseling, feedback and guidance to users looking to manage their medical conditions. These interactions may occur though videos, telephone calls, webinars and others platforms that enable direct and interactive discussions with experts. These types of interactions may be of particular interest in corporate health settings.

Integration within Corporate Health/Providing Incentives

The previously described integrated system may be used as part of larger health and wellness offerings within corporate settings that additionally motivate and incent individuals to adopt health behaviors and lifestyles. While generally described herein as corporate health or wellness, any type of employee/employer relationship or other relationships such as those between individuals and health plans, co-ops, third-party administrators, health plan administrators, human resources outsourcers, labor unions, etc. fall within the scope of the disclosure. Employees (or users) may also include their spouses, partners, children and other dependents on a particular health plan that may be sponsored by a company. Some embodiments of these products and systems may also be used by consumers including patients that have received recommendations from health care providers such as doctors for such programs. Thus the term “employee” may encompass any healthcare plan user, including but not limited to, the types of individual healthcare plan users listed above, or consumers paying out of pocket.

The systems and methods disclosed herein may benefit the employer, employee or user, healthcare insurance provider, and others by improving health and reducing healthcare spend. For example, an employer may save money through improved health and well-being of its employees and reduction in expensive treatments for preventable or chronic health conditions. Similarly, an employee may receive incentives to engage in and improve their health and well being. Additionally, the healthcare insurance provider may recognize a drop in claims submitted, resulting in healthcare cost savings.

FIGS. 24 and 25 show flowcharts of employees in a corporate health environment. In all figures included herein, not every step is required and additional steps may be included. Similarly, the steps do not necessarily need to be performed in the order illustrated. In some embodiments, one or more of the steps may be performed or provided by one or more third-party entities. The systems and methods disclosed herein can be carried out in part by computer programs and in some embodiments, various parts of the systems and methods may be carried out by different entities. For example, data and programs may be stored on one or more remote servers and accessed online by employers and employees over a network, such as the Internet, a LAN (local area network), or WAN (wide area network).

Computing systems for supporting corporate health systems comprise several components including a plurality of computers, smart phones, tablets of other means that are connected with a network such as the internet. The user (or users) can interact with a server in order to input and receive information, to view/update employee profiles, to complete health risk assessments (HRAs) and to view individualized health management solutions that may be customized. There may be a system environment that may include the ability to access one or more web site servers in order to obtain content from the Internet for use with employees' individualized health solution plans and HRAs. The computing system environment may include a plurality of computers and may be scalable to add or delete computers to or from a network.

In general, the computers include a main memory, one or more mass storage devices, a processor, one or more input devices, and one or more output devices. The main memory may include random access memory (RAM), read-only memory (ROM) or similar types of memory. One or more programs or applications such as a web browser and/or other applications may typically be stored in one more data storage devices. Programs or applications may be loaded in part or in whole into main memory or processor during execution by the processor. Mass storage devices may include a hard disk drive, floppy disk drive, CD-ROM drive, smart drive, flash drive or other types of non-volatile data storage, a plurality of storage devices, or any combination of storage devices. The processor may execute applications or programs, stored as executable programs or program code in memory or mass storage device, or received from the Internet or other network. Input devices may include any device for entering information such as a microphone, digital camera, video recorder or camcorder, keyboard, mouse, cursor-control device, touch-tone telephone or touch-screen, a plurality of or any combination of input devices. Output devices may include any type of device for presenting information to a user, including a monitor or flat-screen display (such as tablet/mobile phone), a printer, and speakers or any device for providing information in audio form, and one or more output devices.

Applications such as a web browser may be used to access information for HRAs and individualized health management solutions and display them in web pages, and allow information to be updated. Any web browser or other application capable of retrieving content from a network and displaying pages or screens may be used. In some embodiments, a customized application may be used to access, display and update information for a user to help track adherence over time. Examples of computers include personal desktop computers, laptop computers, notebook computers, network computers, or any processor-controlled device capable of executing a web browser or other type of application for interacting with the system, including mobile devices such as cellular phones.

The server may typically include a main memory, one or more mass storage devices, a processor, one or more input devices, and one or more output devices. Main memory may include RAM, ROM or similar types of memory. One or more programs or applications, such as a web browser and/or other applications, may typically be stored in one or more mass storage devices. Programs or applications may be loaded in part or in whole into main memory or processor during execution by processor. Mass storage devices may include, but are not limited to, a hard disk drive, floppy disk drive, CD-ROM drive, smart drive, flash drive or other types of non-volatile data storage, a plurality of or any combination of storage devices. Processors may execute applications, or portions thereof, stored as executable programs or program code in memory or mass storage device, or received from the Internet or other network. Input and output devices (as previously described) also find use. The server may store a database structure in mass storage device, for example, for storing and maintaining claim data, HRA information, and other outside data. Any type of data structure can be used, such as a relational database or an object-oriented database. Processors may, alone or in combination, execute one or more applications in order to provide some or all of the functions, or portions thereof. Employers may monitor system performance, input data, modify parameters of incentive programs, etc.

In some embodiments, an employee incentive structure and/or health management budget may be calculated for an employer, upon input or information provided by the employer, for example, relating to the employer's goals, size, budget, employee composition, and/or workplace culture. A smaller employer may provide resources toward less expensive health management tools, and lesser incentives, whereas a larger employer may provide more expensive programs and incentives. The particular composition of the workforce may affect the particular incentives and health management programs offered, for instance if a particular workforce has an atypical distribution of age, gender, particular employee health conditions and the like. Workplace culture may affect programs and incentives offered, and incentive and health management programs may be designed to incorporate employee teams and competitions.

As described fully below, a particular incentive structure and budget may be updated and adjusted periodically in response to various factors, including, for example, employee participation, healthcare savings, and/or incentive program costs. The methods and systems of the present disclosure may be implemented as part of a defined contribution health plan, in which employers contribute a fixed amount per employee for healthcare costs, or a traditional fee-for-service health plan, or health plans that have features of both, i.e., employers contribute a part of expected healthcare costs as a defined sum (less, in some cases, an employee's deductible) and the remaining health costs may be paid on a fee-for-service model.

In some embodiments of the present invention such as that shown in FIG. 25, assessing the health of an employee population can be accomplished by an HRA, a health screening and/or biometric screening, by obtaining pharmacy and/or medical claims data, and/or by obtaining data self-reported by the individual.

A HRA may include a set of scientifically validated health and well-being questions that provide an in-depth look at the individual's daily activities. In some cases, a third-party may perform the HRA. However, in other embodiments, any general or customized HRA questions may be used and may vary in complexity and/or length. For example, a HRA may include over 100 questions. Alternatively, a HRA may be generally simple to fill out and include, for example, only a handful of questions. Medically approved algorithms may identify individuals with behavior risks and increased risks for disease. For example, one or more HRA questions may relate to whether an employee has a history of hypertension or whether an employee has common symptoms of hypertension, and may include questions that ask the employee to rate their frequency and/or severity. An algorithm may identify the employee as having high blood pressure or have a risk for developing heart disease, if any of the following conditions are true, based on the employee's answers: (1) the employee has a history of hypertension; (2) the employee has 2 or more symptoms of hypertension; (3) the employee has any hypertension symptom over a certain severity. Other algorithms may be used to identify employees with hypertension, prehypertension, or other health conditions.

A HRA may also gather in-depth information relating to the person's demographics and personal history, family health history, self-care, personal health, women's health, nutrition, physical activity, alcohol and tobacco use, and ability to change behavior. An HRA may be completed by paper questionnaire or online means using software loaded into the main memory or one or more mass storage device of an individual's computer, using voice recognition or touch-tone inputs, by formal interview in person or via a telephone, or other voice communication device, etc. In many cases, the data transfer and storage will be done in a HIPAA compliant manner.

Questions in an HRA may include questions that may elicit biometric information such as age, gender, height, weight, inches around the wrist between the wrist bone and hand, inches around the waist at belly button, inches around the neck, and body frame, etc. Questions may elicit body mass index (BMI), blood pressure, and/or cholesterol, serum triglyceride and glucose concentrations. In other embodiments, some or all of this information may be obtained or updated through health and/or biometric screening.

Other questions that may be asked in a HRA may include an employee's education and/or job function, the employee's family history of cancer, diabetes, heart problems, high blood pressure, high cholesterol, or stroke; whether the employee has a health condition such as allergies, angina, asthma, back pain, cancer, chronic bronchitis/emphysema, depression, diabetes, heart disease, high cholesterol, hypertension, kidney disease, liver disease, migraines, osteoporosis, past stroke, or a thyroid condition; whether medication is being taken to treat a health condition. Health screening, for example, administering one or more medical tests (which may or may not be invasive) to obtain medical data about individual employees, may be used to gather current information relating to one or more of cholesterol levels, diabetes, blood pressure, and other medical information. Relevant medical tests may include for example but not limited to, lipid tests to screen for blood cholesterol levels, blood glucose measurements to screen for diabetes, blood pressure measurements, as well as nicotine levels, PSA (prostate-specific antigen), skin cancer tests, bone density tests, and others. Biometric screening, that is, height, weight, body mass index (BMI) and other such outwardly measurable physiological traits of individual employees may also be used. In one embodiment, a third-party may be used to perform the health screening.

Outside data sources that may be used to gather additional employee health information may include medical and pharmacy claims data from health insurance providers. Outside data sources may include information from an employee or individual's doctor or other healthcare providers, employer or third-party medical and pharmacy claims analyses, as well as self-reported data.

Health data collected from a HRA, PAM, health and/or biometric screening, and/or claims data may be used to generate a personal health score (which may be presented as a report which reveals major threats to the employee's health, how the employee's health history impacts their score, and personalized recommendations based on age, gender, personal history, calculated health risks or readiness for change. A personal health score may be updated after the user incorporates changes in behavior, there is updated health information, etc.

In some embodiments, employees may be identified who have health conditions or lifestyle behaviors that could be addressed through coaching services, health programs, education or other means. Employees may be stratified into intervention intensity levels for each condition/risk factor they have which may be used to tailor coaching services, health programs, education, etc or to increase or decrease dose or frequency of dosing for a dietary supplement for example.

In some cases, an individualized health management solution may contain one or more of the following categories of information for an employee: instructions, teaching employees how to use an individualized health management plan through overviews and tutorials; actions to take, providing a list of recommended health activities and steps that highlight specific behaviors and reward opportunities; education, providing information, articles, quizzes and tools specific to the health needs of each employee; personally information based on each employee's current health situation, including recommended preventative care, and reminders on family history; health programs, outlining eligible programs, including goal setting and tracking; and health records, storing and tracking health statistics, and personal health information, including, in some embodiments, a personal health score; a recommended dietary supplement regimen. An individualized health management solution may also display a summary of information gathered from a HRA, health and/or biometric screening or other sources, such as claims data. Further, an individualized health management solution may provide several available options, such as exercise plans, health coaching, scheduling preventative check-ups, etc. for an individual to begin a path to better health.

Individualized health management solutions may facilitate or incorporate various health coaching programs. Health coaching may include online training, telephone conferences, or other interactions with a health coach for one or more of an employee's conditions. An individualized health management solution may provide meeting reminders, self-directed and other educational materials designed to facilitate and supplement health coaching. Health coaching may involve self-directed programs, which may be accessed through an online and may include a nutritional guide, which may suggest simple, convenient meal ideas, recipes and tips to help employees eat healthier. In further embodiments, programs may also include organizing and tracking online team building, i.e., organizing a plurality of employees interested in achieving similar health goals, and organizing and tracking workplace health competitions, in which incentive rewards are given to an individual employee or team who achieves a certain health goal.

Coaching and health programs may further involve live health and wellness ‘webinars’; coach-customer ‘personality’ matching using employee profiles and other data; incorporation of remote health monitoring technology, such as step counters or pedometers, blood pressure monitors, etc. In some embodiments a health program may include a fitness activity tracker, in which employees are encouraged to incorporate daily physical activity. One goal may be to encourage an individual to achieve at least 30 minutes, or other suitable amount, of daily physical activity as evidenced by a certain number of steps (ie approximately or greater than 1000, 2000, 3000, 4000, 5000, 6000, 7000, 8000, 9000, 10000, 11000, 12000, 13000, 14000, 15000, 16000, 17000, 18000, 19000, 20000 or more). Employees may enter their daily activity data online, and the personal fitness activity tracker may automatically calculate or estimate the number of calories burned as a result of exercise. Daily activity may also be recorded by an employee through other means, such as in a paper journal. A fitness activity tracker may provide nutritional advice, including meal plans, as well as continual program progress, tips, and other motivational support. In some embodiments, an individual's health management solution may allow the individual to input new conditions, update improving or worsening conditions, and track improvement. An individual may directly input updated biometric information such as weight loss or gain, etc.; updated health screening information, such as cholesterol levels, etc.; and daily activity information, such as steps, exercises performed, etc.

Incentives may be used as an inducement for employees to participate in a HRA and/or health or biometric screening and may be used to help induce employees to monitor and follow their personal health solution and/or engage in health management. Incentives provided can be tailored to both an individual employer and employee, and may include, but are not limited to, an offset in an individual's contribution to his/her health plan, deposits into an HRA, 401(k), cash, points, paid time-off hours, and other special privileges available to all qualifying individuals, etc. Incentive-earning behaviors can be entered into an incentive program system online by an employee or others. Employee entries can be periodically audited, automatically approved, or approved after review and/or validation. Employers can determine time limits or expiration dates of incentives for achieving a particular health result. Incentives can be distributed to employees yearly, quarterly, on a task-complete basis, upon request for distribution, or at any other suitable timeframe.

An employee may participate in a health screening program, and data collected may include height, weight measurements; BMI; blood pressure; total cholesterol; HDL, LDL; triglycerides; and blood glucose, etc. Results may be shared with the employee and discussed with a health educator. Data collected may also be sent to a data server, where it can be collected and stored, for example, in mass storage device. An employee may also complete a HRA and may receive results and feedback, including the calculation of a personal health score, a health potential score, and options presented to improve a personal health score. Data from the HRA and/or health scores may be sent to a server, where it can be collected and stored. An employee may need to complete one or more steps to receive an incentive, however, in some embodiments separate incentives may be granted upon completion of each step or mere participation in the program.

Self-directed health programs, preventative care, and/or a personal health coaching programs listed in may involve disease, health conditions or lifestyle management programs. As an example, an employee may select a walking program to boost activity levels and lose weight. The employee may be required to complete and submit a tracking log or other tracking mechanism, such as entering information using a physical activity tracker, be actively engaged for 6 months, or other suitable time period, in the program; achieve a specified average number of steps per day (e.g., for 5 days/wk.)

Incentives may also be given for completing health coaching programs. Employees may be placed into appropriate health coaching programs over the telephone, web (via sound, video and/or text-based coaching services); or in print, etc. Types of health coaching may include behavior change coaching, condition management coaching, and specialty condition management coaching. Behavior change coaching may involve helping to increase physical activity, managing obesity, coping with stress, improving nutrition, lowering tobacco use, lowering high blood pressure or cholesterol level, etc. Condition management coaching may involve managing diabetes, coronary artery disease, pregnancy, or chronic back pain. Specialty condition management coaching may involve helping cope with surgeries.

Incentives may be provided to an individual for meeting one or more health metric standards such as those for BMI, blood pressure, total cholesterol, HDL, LDL, triglycerides, blood glucose level, lowered tobacco use, etc. An incentive program may also involve incentives received through team efforts or team goal setting, in which incentives are given based on the performance of a plurality of team members toward a goal, such as weight loss or increased exercise.

Incentives may be awarded at any appropriate time. For example, incentives may be awarded immediately upon enrollment, during participation of, or at completion of a health-related task or program, such as a health screening, coaching program, or quitting smoking, etc. In other embodiments, the award of incentives may be delayed until a point in time after completion of a health-related task or program or phased-in upon reaching one or more milestones during participation of a health-related task or program, such as 1) at enrollment, 2) at a mid-point of participation, and 3) at program completion.

User Experience

There are at least two use cases. In the first, the user is an employee in a company that has a health and wellness program. The user may have an annual checkup (and may be incented to have such a checkup) in which a third party provider comes to his or her workplace to measure his blood pressure and do a blood draw. The user is then informed that he or she has a medical condition or pre-condition (such as borderline high cholesterol, high cholesterol, prehypertension and hypertension as examples.) This information may be provided to the individual by the third party that completed the blood draw, a separate third party, or in some cases, the company itself may alert the individual.

The user may then opt to go to a website that has a “storefront” for products that could treat or otherwise manage the medical condition. In this case, the user may choose the integrated product described herein that manages cholesterol and the sponsor company could pay for all or a portion of the cost of the product on an ongoing basis. The user would then receive a weekly, monthly, or longer, supply of the product and would be instructed to take a daily quantity of the dietary supplements in addition to being exposed to the behavior change messaging.

In some cases, the user would consume a full packet of dietary supplements (for example 1, 2, 3, 4, 5, 6, 7, 8, 9, 10, etc. pills, tablets, softgels, gummies) per day in one sitting or over two or more sittings during the day. For example, the user may eat three gummies (to manage cholesterol) within two hours of breakfast, and then another three gummies within two hours of lunch or dinner. Each packet of dietary supplement would have one or more behavior change messages on the pouch itself that will enable the user to treat that condition, or otherwise become more healthy.

The messages on the pouch would be similar to messages contained on an accompanying app (or website designed for mobile or computer use) present on the user's phone or computer. The user would receive one or more notifications during the day to track which healthy behaviors the user completed that day. For example, in the case of cholesterol management, there may be 6-7 healthy behaviors promoted on the pouch materials. The app would provide user education (and would link to videos) and would present how well the user was adhering to the behavior change principles, providing graphic depiction of how many points the user had accumulated over a multi day or weekly period. The user could watch the videos and learn about why some behaviors were positive, and why others were negative. The user might earn additional points for watching one or more videos.

The user may then receive a gift card, cash or other incentive if he or she earned enough points on the app or achieved a certain goal such as a blood pressure reduction of 5 or 10 points, or a reduction in cholesterol of LDL cholesterol of 10% or 10 points for example.

The overall goal of the program is to help create a routine set of behaviors (including taking the supplement) with the goal of helping to maintain health and/or treat various medical conditions or pre-conditions. All components of the system are aimed at creating this routine which is important to ensure the behavior change is sustainable, thereby optimizing the maintenance of good health and disease prevention. The combination of the various components is expected to offer synergistic improvements beyond those from each of the individual components.

Model of Engaging User and Creating Daily Routine

The following example is for a user hoping to maintain healthy levels of blood cholesterol. In the morning, the user has breakfast, ideally comprising oats (or other foods with high levels of dietary fiber and low saturated fat). The user then takes his first dose of plant sterols, having removed the plant sterol gummy from the pouch. He is reminded by the pouch to avoid unhealthy snacks during the day (such as those with high saturated fat or excess sugar) and to replace them with nuts. He takes a small packet of nuts to work with him. As he arrives at work, he remembers his daily goal of walking 7500 steps and decides to walk up the stairs instead of taking the elevator. At lunch time, he takes his second dose of plant sterols for the day, and is again reminded by the pouch to minimize saturated fat and excess sugar. As a result, instead of having his “normal” cheeseburger, French fries and soda, he opts for a turkey sandwich, baked potato chips and a glass of water. Later in the afternoon, he skips his routine candy bar snack and has a bag of almonds he had brought from home. On his way home, he stops by the gym for a light workout and follows that up with a lighter than normal dinner. In the evening, he checks his step count for the day. At 8 pm, he receives a push notification from the app and he “checks off” all the healthy behaviors for the day. In total, he has earned 8 of a total of 14 points that day. He decides to watch a short video that he links to directly from the app to learn more about fiber intake. As a result of these simple interventions, he ate 1000 less calories during the day and expended 400 more calories than normal.

What is claimed is:

1. An integrated disease management system for enhancing a user's health, the system comprising:
an outer box;
six or more pouches within the outer box;
a dietary supplement within each of the pouches;
wherein each pouch comprises a front, having a first panel, and a back having a second panel, and wherein the first panel of each pouch includes an educational message that is different from the educational message on any of the other pouches.

2. The system of claim 1, further comprising a non-transitory computer-readable storage medium storing a set of instructions capable of being executed by a smartphone, that, when executed by the smartphone, causes the smartphone to: receive input of user activities including: user consumption of a dietary supplement, user physical activity, and user food consumption; score the inputted user activities; track scores over time; and display one or more representations of user progress based on the scores.

3. The system of claim 2, further comprising a remote server executing a second set of instructions capable of being executed by a processor of the remote server, the second set of instructions that, when executed by the processor, causes the server to receive user scores and to indicate distribution of a reward to a user based on the received user scores.

4. The system of claim 2, further comprising a library of videos directed to health and wellness topics, further wherein the set of instructions causes the smartphone to access the library of videos for display to the user.

5. The system of claim 1, wherein the dietary supplement comprises a pill, tablet, gummy, powder, softgel, chew, capsule, or gel.

6. The system of claim 1, wherein each pouch comprises at least two sealed chambers, wherein each chamber includes the dietary supplement.

7. The system of claim 1, wherein the dietary supplement comprises a gummy.

8. The system of claim 1, wherein the dietary supplement comprises a phytosterol.

9. The system of claim 1, wherein the non-transitory computer-readable storage medium is further configured to transmit user scores to a remote server.

10. The system of claim 9, wherein the remote server creates customized content based on received input of user activities and transmits the customized content to the smartphone for display to the user.

11. The system of claim 1, wherein the back comprises the second panel and a third panel that are adjacent to each other when the pouch is closed, and a fourth panel between the second and third panels that is exposed only when the pouch is opened, to reveal hidden text or image related to the educational message.

12. An integrated disease management system for enhancing employee health, the system comprising:
an outer box;
a plurality of closed pouches within the outer box;
a dietary supplement within each of the plurality of pouches;
and a non-transitory computer-readable storage medium storing a set of instructions capable of being executed by a smartphone, that, when executed by the smartphone, causes the smartphone to: receive input of user activities including user consumption of the dietary supplement; and display one or more representations of user progress based on the inputted user activities; and
a library of videos directed to health and wellness topics.

13. The system of claim 12, wherein the dietary supplement comprises a pill, tablet, gummy, powder, softgel, chew, capsule, or gel.

14. The system of claim 12, wherein each pouch comprises at least two sealed chambers, wherein each chamber includes the dietary supplement.

15. The system of claim 12, wherein the dietary supplement comprises a gummy.

16. The system of claim 12, wherein the dietary supplement comprises a phytosterol.

17. The system of claim 12, wherein each pouch further comprises a hidden text or image related to the educational message that is revealed only upon opening the pouch.

18. A method of improving a user's health by multiple daily messaging encouraging routine healthy behaviors, the method comprising:
providing a plurality of pouches, wherein each of the plurality of pouches comprises a dietary supplement, further wherein each pouch comprises an educational message;
providing a non-transitory computer-readable storage medium storing a set of instructions capable of being executed by a smartphone, that, when executed by the smartphone, causes the smartphone to: receive input of user activities including user consumption of the dietary supplement and one or more of: user physical activity and user food consumption; score the inputted user activities;
track scores over time; display one or more representations of user progress based on the scores; and transmit the received input of user activities or scores to a remote server;
receiving, in the remote server, the input of user activities or scores; and
tracking, in the remote server, user consumption of the dietary supplement and/or user activity.

19. The method of claim 18, further comprising: delivering customized educational content to the user based on the input of user activities or scores.

20. The method of claim 18, further comprising connecting the user to a medical professional based on the input of user activities or scores.

21. The method of claim 18, further wherein the set of instructions causes the smartphone to transmit the received input of user activities or scores to a remote server using encryption to protect the user's privacy.
